(19) United States
(12) Patent Application Publication
(10) Pub. No.: US 2026/0232942 A1
(43) Pub. Date: Aug. 13, 2026

(54) MEDICAL TUBES FOR A BREATHING CIRCUIT

(71) Applicant: Fisher & Paykel Healthcare Limited, Auckland (NZ)

(72) Inventors: Samuel James STOCKWELL, Auckland (NZ); Ashley VAN STADEN, Auckland (NZ); Karla Maree DEY, Auckland (NZ); Paul Joseph MOODY, Auckland (NZ); Telge Nishan Chaturanga PEIRIS, Auckland (NZ); Theodore Hamilton Robertshawe PEPPER, Auckland (NZ); Geoff Kok-Hoe ONG, Auckland (NZ)

(21) Appl. No.: 19/112,209

(22) PCT Filed: Sep. 29, 2023
(86) PCT No.: PCT/IB2023/059735

§ 371 Date: Mar. 14, 2025

Related U.S. Application Data
(60) Provisional application No. 63/377,991, filed on Sep. 30, 2022.

(51) Int. Cl. A61M 16/10; A61M 16/00; A61M 16/08; A61M 16/16

(52) U.S. Cl. CPC A61M 16/1095; A61M 16/0841; A61M 16/0875; A61M 16/16; A61M 2016/003; A61M 2205/3368; A61M 2240/00

(57) ABSTRACT

This disclosure relates to a medical tube for humidified gas; comprising first and second tube segments each having a heater wire. The electrical power used by the second heater wire can be determined by a property of the first heater wire and/or second heater wire. The disclosure relates to a medical tube comprising multiple tube segments that are configured to provide different levels of heating. The tube heaters are controlled by a single controller, and only one tube segment is directly electrically connected to the controller such that the downstream tube segment only receives electrical power via the upstream tube segment. The ratio of the power delivered to the first tube segment and to the second tube segment can be configured by one or more characteristics of one or both tube segments.

### BACKGROUND

### Field

This disclosure relates generally to medical tubes, suitable for medical use, and in particular, to medical tubes for use in a breathing circuit suitable for providing humidified gas to a patient and/or removing gas from a patient, such as in respiratory humidification systems. The disclosed medical tubes can in some cases have other applications, for example in surgery, such as laparoscopy. This application claims priority from U.S. provisional application 63/377,991 filed on 30 Sep. 2022, the entire contents of which are herein incorporated by reference.

### Description of Related Art

In breathing circuits, various components transport breathable gases which may be warm and/or humidified, to and from patients. For example, medical tubes may be used in respiratory humidification systems to convey respiratory gases between a respiratory component, such as a ventilator or a humidifier, and a patient.

Breathable gases can be heated and/or humidified prior to delivery to the patient to mimic the transformation of air that occurs as it enters the respiratory system. Heated medical tubes can deliver the heated and/or humidified respiratory gases directly to a patient interface or, in some cases, an additional medical tube can be located between the heated medical tube and the patient interface. Respiratory humidification can help reduce the likelihood of infection and/or tissue damage.

An example medical tube is as described in our earlier international application WO2012154064 having an earliest priority date of 6 May 2011 and published on 15 Nov. 2012, in our earlier international application WO2018217105 having a priority date 26 May 2017 and published on 29 Nov. 2018, and in our earlier international application WO2019/164409 having a priority date of 23 Feb. 2018 and published on 29 Aug. 2019. Medical tubes can be used with an incubator, for example as described by our earlier international application WO2014/077706 having an earliest priority date of 14 Nov. 2012 and published on 22 May 2014. The entire contents of each of these earlier applications are hereby incorporated by reference in their entirety.

### SUMMARY

Medical tubes can be used in breathing circuits of respiratory therapy systems, for example, for delivering and/or removing humidified gases from a patient, such as in obstructive sleep apnea, neonatal, respiratory humidification, and/or in surgical humidification systems including insufflation systems, and in systems for patients undergoing procedures under general anesthetic.

Medical tubes can typically comprise a number of different components that are assembled together.

Medical tubes can comprise an end connector at one or both ends of the tube. The end connector enables the tube to be connected to other components, for example to another tube, a patient interface, a humidifier, or an adaptor. The end connector may be configured to incorporate or receive one or more sensors.

Medical tubes can comprise one or more wires, extending along some or all of the length of the tube.

Medical tubes can be reprocessed so as to be able to be reused. Reprocessing of medical tubes can include cleaning and/or sterilisation of the medical tubes, for example using an autoclaving process.

The medical tubes described herein can include a substantially smooth bore formed by a bead and film.

In some examples, a medical tube for transporting gases can comprise a bead spirally wrapped around a longitudinal axis of the medical tube. The bead can form a first portion of a lumen wall of the medical tube. The medical tube can also comprise a film spirally wrapped around the longitudinal axis of the medical tube. A first portion of the film may overlie the bead and a second portion of the film may form a second portion of the lumen wall. The lumen wall, formed by the bead and the second portion of the film, can comprise a substantially smooth bore of the medical tube.

In some embodiments, the medical tube can include one or more of the following features, in any combination:
a) The bead can comprise a heating element. The heating element can comprise one or two or more heater wires disposed within the bead. Reference in this disclosure to a ‘heating wires’ includes reference to multiple, separate wires, and also to a single heating wire that may be looped back on itself to form two wire portions.
b) The bead can comprise at least one sensor and/or data wire for conveying power and/or data between at least one sensor and a controller. The at least one sensor wire can comprise one or two or more sensor wires disposed within the bead. The at least one sensor can comprise at least one of a temperature sensor, a humidity sensor, a flow sensor, and a pressure sensor.
c) The bead can comprise one or more ground or earth wires.
d) The bead can comprise a substantially flat surface forming the first portion of the lumen wall. The second portion of the film can comprise a substantially flat surface forming the second portion of the lumen wall.

According to an aspect of this disclosure there is provided a medical tube for transporting a humidified gas; comprising
a first tube segment comprising:
   a first lumen for transporting the humidified gas; and
   a first heater wire;
a second tube segment comprising:
   a second lumen for transporting the humidified gas; and
   a second heater wire; wherein
the first and second tube segments are configured such that:
   the first lumen is pneumatically connected to the second lumen; and the first heater wire is electrically connected to the second heater wire; wherein
the first heater wire is configured to be electrically connected to, and to use electrical power provided by, a power source; and
   wherein the second heater wire also uses the electrical power, wherein the electrical power used by the second heater wire is determined by a property of the first heater wire and/or by a property of the second heater wire.

The property of the first and/or second heater wires may be an electrical property.

The electrical property may be the electrical resistance of the wire. The resistance is calculated with reference to the resistivity of the wire material(s) used.

The electrical resistance may be determined by one or more physical properties of the first and/or second heater wires, wherein the physical properties include any one or more of:
a) resistivity of the wire material(s);
b) wire gauge, that is, wire diameter;
c) wire material;
d) wire length;
e) if the wire is helically wound, any one or more of:
   i. the helix pitch;
   ii. the helix angle;
   iii. the helix length;
   iv. the helix diameter;
   v. the number of helix windings.

The helix length, as referred to in this specification, means the axial length of the tube (or portion of the tube) along which the wire is helically wound.

The electrical power used by the second heater wire may be determined by one or more physical properties of the medical tube.

The electrical resistance of the wire may be determined by one or more physical properties of the medical tube.

The physical property may be any one or more of the following:
a) tube segment material;
b) tube segment length;
c) tube segment inner diameter (i.e. lumen diameter);
d) tube segment insulation;
e) tube segment bead material;
f) tube segment bead diameter;
g) tube segment bead length;
h) tube segment bead helix pitch;
i) tube segment bead helix angle;
j) tube segment bead helix length;
k) the number of tube segment helix windings.

The properties of the first and second tube segments may be configured such that between 3-20%, and optionally 3-12%, of the total electrical power delivered to the inspiratory circuit by the power source is used by the downstream second tube segment.

The medical tube may comprise any one or more of the following characteristics:
a) the length of the first inspiratory segment is between 1100-1400 mm, optionally 1300 mm;
b) the length of the second inspiratory segment is between 200-400 mm, optionally 300 mm;
c) the diameter of the wire in the first inspiratory segment is between 0.1 and 0.3 mm, optionally approximately 0.2 mm;
d) the diameter of the wire in the second inspiratory segment is between 0.2 and 0.5 mm, and optionally approximately 0.3 mm;
e) the resistance of the heater wire of the inspiratory circuit is between 10 and 25Ω, and optionally is approximately 17Ω;
f) the resistance of the heater wire of the first inspiratory segment may be between 10 and 25Ω, and optionally is approximately 15Ω;
g) the resistance of the heater wire of the second inspiratory segment may between 0.5 and 3Ω, and optionally is approximately 1Ω.

The first tube segment comprises a length, wherein the length may be:
a) at least about 0.5 m and/or less than or equal to about 2 m;
b) at least about 0.7 m and/or less than or equal to about 1.8 m;
c) at least about 0.9 m and/or less than or equal to about 1.5 m;
d) at least about 1 m and/or less than or equal to about 1.2 m; or
e) at least about 1.2 m and less than or equal to 1.3 m.

The second tube segment comprises a length, wherein the length may be:
a) at least about 0.2 m and/or less than or equal to about 1.5 m;
b) at least about 0.3 m and/or less than or equal to about 1 m;
c) at least about 0.4 m and/or less than or equal to about 0.8 m;
d) at least about 0.5 m and/or less than or equal to about 0.7 m; or
e) at least about 0.25 m and/or less than 0.35 m, and optionally at least about 0.28 m and/pr less than 0.32 m.

The heater wires in each tube segment may be configured such that one of the heater wires comprises:
a) the same wire diameter but different resistance values from the other heater wire;
b) different wire helix pitch from the other heater wire;
c) different wire diameter and different resistance values from the other heater wire; and/or
d) different wire diameter but the same resistance valve from the other heater wire.

One or both tube segment may comprise a plurality of heater wires.

The heater wire of one or both tube segment may comprise a variable wire helix pitch along some or all of the length of the tube segment.

The wire helix pitch at the patient end of the second tube segment may be configured to increase the flexibility of the second tube segment over a portion of its length, as compared to the flexibility along the remainder of the length of the second tube segment.

The flexibility of one tube segment may be different to that of the other tube segment. The flexibility may be determined by one or more physical properties of the tube segment.

The physical property may be any one or more of the following:
a) tube segment material;
b) tube segment length;
c) tube segment inner diameter (i.e. lumen diameter);
d) tube segment insulation;
e) tube segment bead material;
f) tube segment bead diameter;
g) tube segment bead length;
h) tube segment bead helix pitch;
i) tube segment bead helix angle;
j) tube segment bead helix length;
k) the number of tube segment helix windings.

The heater wire of one tube segment may be wound at a different winding density to that of the other tube segment.

The first and second tube segments may be electrically connected in series.

The first and second tube segments may always use electrical power at the same time.

The second tube segment may be only electrically connected to the first tube segment, that is, the second tube segment is not directly electrically connected to the power source.

The first and second tube segments may be configured to each use the same current when provided with electrical power by the power source.

The medical tube may comprise at least one intermediate connector, wherein the first and second lumens are connected together via the at least one intermediate connector. The at least one intermediate connector may be configured to:
a) pneumatically connect the first lumen to the second lumen; and/or
b) electrically connect the first heater wire to the second heater wire.

The medical tube may comprise a plurality of intermediate connectors. One intermediate connector may pneumatically connect the first lumen to the second lumen. One intermediate connector may electrically connect the first heater wire to the second heater wire.

Alternatively one intermediate connector may comprise a plurality of sub-connectors. One sub-connector may pneumatically connect the first lumen to the second lumen. One sub-connector may electrically connect the first heater wire to the second heater wire.

The intermediate connector may comprise an electrical connector.

The intermediate connector may comprise a probe port configured to receive a probe to measure a property of the humidified gas. The probe optionally comprises a temperature probe.

The medical tube may comprise a device end connector configured to connect the first tube segment to a device providing a flow of the humidified gas.

The device end connector may comprise a probe port body comprising a probe port configured to receive a probe to measure a property of the humidified gas, the probe optionally comprising a temperature probe and/or a flow probe. The probe may comprise a temperature sensor and/or a flow sensor.

The device end connector may comprise an electrical connector configured to be connected to a device comprising the power source.

The intermediate connector may comprise a rigid connector body comprising a lumen for transporting the humidified gas, the lumen comprising a longitudinally extending lumen axis.

The intermediate connector may comprise an overmould that encapsulates some or all of the rigid connector body

The overmould may extend beyond the ends of the rigid connector body.

The overmould may extend over the first and/or second tube segments.

The overmould may pneumatically connect the first and/or second tube segments to the rigid connector body.

The overmould may extend at least partially over the probe port body.

At least one of the first and second tube segments may comprise a spirally wound body having a helix angle; and wherein the connector body may comprise an end face adjacent the end of the at least one tube segment;
wherein the end face comprises a helix angle that is substantially identical to the helix angle of the spirally wound body; and/or wherein
the end face comprises an offset portion configured to receive a portion of the spirally wound body, the offset portion being offset in a direction along the axis of the lumen of the tube by a distance substantially identical to the pitch of the spirally wound body and/or the width of the spirally wound body.

The offset portion may at least partially retain a portion of the spirally wound member.

The offset portion may comprise a channel configured to at least partially receive the spirally wound body.

The channel may comprise an upstanding wall against which the spirally wound body abuts, when received in the channel.

The channel may comprise a recess, recessed radially inwardly in a direction towards a longitudinal axis of the connector body, relative to an adjacent portion of the connector body.

The end face may be at least partially spaced along the axis of the lumen from the end of the at least one tube segment.

The medical tube may comprise a pair of electrical conductors on the rigid connector body, each electrical conductor being configured to electrically connect with a respective heater wire.

The first heater wire may extend at least partially around the exterior of the rigid connector body in a first direction, from the first tube segment to the first electrical conductor, and the second heater wire may extend at least partially around the exterior of the rigid connector body in a second, opposite, direction, from the second tube segment to the second electrical conductor.

One of the first and second heater wires may be wound around the rigid connector body in a clockwise direction, when the rigid connector body is viewed from a first end of the rigid connector body along a longitudinal axis extending through the lumen of the rigid connector body, the other of the first and second wires being wound around the rigid connector body in an anti-clockwise direction when the rigid connector body is viewed from the first end of the rigid connector body along a longitudinal axis extending through the lumen of the rigid connector body.

The wires may extend around between 25% and 75% of the periphery of the rigid connector body, when viewed along the lumen axis.

The exterior of the rigid connector body may be arcuate.

The rigid connector body may be circular when viewed along the lumen axis.

The exterior of the rigid connector body may be substantially cylindrical.

The medical tube may be an inspiratory tube for transporting breathable gases.

The medical tube may be a surgical tube for transporting surgical gases. The medical tube may be a laparoscopic tube.

According to another aspect of this disclosure there is provided a medical tube for transporting a humidified gas; comprising
a first tube segment comprising a first lumen for transporting the humidified gas, and a first heater wire;
a second tube segment comprising a second lumen for transporting the humidified gas, and a second heater wire;
   wherein the first heater wire is configured to be electrically connected to, and to use electrical power provided by, a power source;
wherein the second heater wire also uses the electrical power;
wherein the proportion of the total electrical power used by the second heater wire is between 3 and 20% of the total electrical power used by the first and second tube segments.

According to another aspect of this disclosure there is provided a medical tube for transporting a humidified gas; comprising
a first tube segment comprising a first lumen for transporting the humidified gas, and a first heater wire;
a second tube segment comprising a second lumen for transporting the humidified gas, and a second heater wire; wherein
the first tube segment is configured to be electrically connected to, and to use electrical power provided by, a power source; and
wherein the second heater wire also uses the electrical power; wherein the first heater wire has a higher resistance value than that of the second heater wire.

According to another aspect of this disclosure there is provided a medical tube for transporting a humidified gas; comprising
a first tube segment comprising a first lumen for transporting the humidified gas, and a first heater wire;
a second tube segment comprising a second lumen for transporting the humidified gas, and a second heater wire; wherein
   the first tube segment is configured to be electrically connected to, and to use electrical power provided by, a power source; and
wherein the second heater wire also uses the electrical power; and wherein the first heater wire determines the electrical power used by the second heater wire.

The first and/or second heater wires may comprise a property that determines, or partially determines the electrical power used by the first and/or second heater wires.

The first and/or second heater wires may comprise a property that determines, or partially determines the resistance of the first and/or second heater wires.

The property of the first and/or second heater wires may be an electrical property.

The electrical property may be electrical resistance.

The electrical resistance may be determined by one or more physical properties of the first and/or second heater wires, wherein the physical properties include any one or more of:
a) resistivity of the wire material(s);
b) Wire gauge, that is, wire diameter;
c) Wire material;
d) Wire length;
e) If the wire is helically wound:
   a) The helix pitch.
   b) The helix angle.
   c) The helix length.
   d) The helix diameter.
   e) The number of helix windings.

The electrical power used by the first and/or second heater wires may be determined by one or more physical properties of the medical tube.

The physical property may be any one or more of the following:
a) Tube segment material;
b) Tube segment length;
c) Tube segment inner diameter (i.e. lumen diameter);
d) Tube segment insulation;
e) Tube segment bead material;
f) Tube segment bead diameter;
g) Tube segment bead length;
h) Tube segment bead helix pitch;
i) Tube segment bead helix angle;
j) Tube segment bead helix length;
k) The number of tube segment helix windings.

The properties of the first and second tube segments may be configured such that between 3-20%, and optionally 3-12%, of the total electrical power delivered to the inspiratory circuit by the power source is used by the downstream second tube segment.

The medical tube may comprise any one or more of the following characteristics:
a) The length of the first inspiratory segment is between 1100-1400 mm, optionally 1300 mm.
b) The length of the second inspiratory segment is between 200-400 mm, optionally 300 mm.
c) The diameter of the wire in the first inspiratory segment is between 0.1 and 0.3 mm, optionally approximately 0.2 mm.
d) The diameter of the wire in the second inspiratory segment is between 0.2 and 0.5 mm, and optionally approximately 0.3 mm.
e) The resistance of the heater wire of the inspiratory circuit is between 10 and 25Ω, and optionally is approximately 17Ω.
f) The resistance of the heater wire of the first inspiratory segment may be between 10 and 25Ω, and optionally is approximately 15Ω.
g) The resistance of the heater wire of the second inspiratory segment may between 0.5 and 3Ω, and optionally is approximately 1Ω.

The first tube segment comprises a length, wherein the length may be:
a) at least about 0.5 m and/or less than or equal to about 2 m;
b) at least about 0.7 m and/or less than or equal to about 1.8 m;
c) at least about 0.9 m and/or less than or equal to about 1.5 m; or
d) at least about 1 m and/or less than or equal to about 1.2 m.

The second tube segment comprises a length, wherein the length may be:
a) at least about 0.2 m and/or less than or equal to about 1.5 m;
b) at least about 0.3 m and/or less than or equal to about 1 m;
c) at least about 0.4 m and/or less than or equal to about 0.8 m;
d) at least about 0.5 m and/or less than or equal to about 0.7 m; or
e) at least about 0.25 m and/or less than 0.35 m, and optionally at least about 0.28 m and/or less than 0.32 m.

The heating wires in each tube segment may be configured such that one of the heating wires comprises:
a) the same wire diameter but different resistance values from the other heating wire;
b) different wire helix pitch from the other heating wire;
c) different wire diameter and different resistance values from the other heating wire; and/or
d) different wire diameter but the same resistance valve from the other heating wire.

One or both tube segment may comprise a plurality of heater wires.

The heater wire of one or both tube segment may comprise a variable wire helix pitch along some or all of the length of the tube segment.

The wire helix pitch at the patient end of the second tube segment may be configured to increase the flexibility of the second tube segment over a portion of its length, as compared to the flexibility along the remainder of the length of the second tube portion.

The flexibility of one tube segment may be different to that of the other tube segment.

The heater wire of one tube segment may be wound at a different winding density to that of the other tube segment.

The first and second tube segments may be electrically connected in series.

The first and second tube segments may always use electrical power at the same time.

The second tube segment may be only electrically connected to the first tube segment, that is, the second tube segment is not directly electrically connected to the power source.

The first and second tube segments may be configured to each use the same current.

The medical tube may further comprise an intermediate connector, wherein the first and second lumens are connected together via the intermediate connector.

The intermediate connector may comprise a probe port configured to receive a probe to measure a property of the humidified gas, the probe optionally comprising a temperature probe and/or a flow probe. The probe may comprise a temperature sensor and/or a flow sensor.

The medical tube may comprise a device end connector configured to connect the first tube segment to a device providing a flow of the humidified gas.

The device end connector may comprise a probe port configured to receive a probe to measure a property of the humidified gas, the probe optionally comprising a temperature probe and/or a flow probe. The probe may comprise a temperature sensor and/or a flow sensor.

The device end connector may comprise an electrical connector configured to be connected to a device comprising the power source.

The intermediate connector may comprise a rigid connector body comprising a lumen for transporting the humidified gas, the lumen comprising a longitudinally extending lumen axis.

The intermediate connector may comprise an overmould that encapsulates some or all of the rigid connector body.

The overmould may extend beyond the ends of the rigid connector body.

The overmould may extend over the first and/or second tube segments.

The overmould may pneumatically connect the first and/or second tube segments to the rigid connector body.

The overmould may extend at least partially over the probe port.

At least one of the first and second tube segments may comprise a spirally wound body having a helix angle; and wherein the connector body comprises an end face adjacent the end of the at least one tube segment;
wherein the end face comprises a helix angle that is substantially identical to the helix angle of the spirally wound body; and/or wherein
the end face comprises an offset portion configured to receive a portion of the spirally wound body, the offset portion being offset in a direction along the axis of the lumen of the tube by a distance substantially identical to the pitch of the spirally wound body and/or the width of the spirally wound body.

The end face may be at least partially spaced along the axis of the lumen from the end of the at least one tube segment.

The medical tube may comprise a pair of electrical conductors on the rigid connector body, each electrical conductor being configured to electrically connect with a respective heater wire.

The first heater wire may extend at least partially around and/or along the exterior of the rigid connector body in a first direction from the first tube segment to the first electrical conductor when the rigid connector body is viewed from a first end of the rigid connector body along a longitudinal axis extending through the lumen of the rigid connector body, and the second heater wire may extend at least partially around and/or along the exterior of the rigid connector body in a second, opposite, direction, from the second tube segment to the second electrical conductor when the rigid connector body is viewed from the first end of the rigid connector body along a longitudinal axis extending through the lumen of the rigid connector body.

One of the first and second heater wires may be wound around the rigid connector body in a clockwise direction, when the rigid connector body is viewed along a longitudinal axis extending through the lumen of the rigid connector body, the other of the first and second heater wires being wound around the rigid connector body in an anti-clockwise direction.

The heater wires may extend around between 25% and 75% of the periphery of the rigid connector body, when viewed along the lumen axis.

At least one heater wire, or at least a portion thereof, may be internal of the spirally wound body, so as to be at least partially encapsulated by the spirally wound body.

At least one heater wire, or at least a portion thereof, may be external of the spirally wound body.

At least one heater wire may comprise a heater wire portion that is external of the spirally wound body, where the heater wire is electrically connected to an electrical connector.

The exterior of the rigid connector body may be arcuate.

The rigid connector body may be circular when viewed along the lumen axis.

The exterior of the rigid connector body may be substantially cylindrical.

The medical tube may be an inspiratory tube for transporting breathable gases.

According to another aspect of this disclosure there is provided an intermediate connector for a breathing limb of a breathing circuit, the breathing limb comprising:
a first tube segment comprising a first lumen, and a first heater wire;
a second tube segment comprising a second lumen, and a second heater wire;
wherein the intermediate connector comprises a connector lumen, the intermediate connector being configured to pneumatically connect the first and second lumens via the connector lumen, and to electrically connect the first and second heater wires;
wherein the intermediate connector comprises a connector body and a probe port extending from the connector body, the probe port being configured to receive a probe to measure a characteristic of gas flowing through the connector lumen;
wherein the probe port, connector body and first and second tube segments are overmoulded.

According to another aspect of this disclosure there is provided a medical tube connector configured to connect to an end of a medical tube;
the medical tube comprising a spirally wound body having a pitch and a helix angle; wherein the connector comprises a connector body having an end face adjacent the end of the medical tube;
wherein the end face comprises a helix angle that is substantially identical to the helix angle of the spirally wound body; and/or
the end face comprises an offset portion configured to receive a portion of the spirally wound body, the offset portion being offset in a direction along the axis of the lumen of the medical tube by a distance substantially identical to the pitch of the spirally wound body and/or the width of the spirally wound body.

According to another aspect of this disclosure there is provided a medical tube connector configured to connect to an end of a medical tube;
the medical tube comprising a spirally wound body; wherein the connector comprises a connector body;
wherein the connector body comprises an offset portion configured to receive a portion of the spirally wound body, the offset portion being offset in a radially inward direction towards the axis of the lumen of the medical tube.

The offset portion of the connector body may comprise an end face adjacent the end of the medical tube.

The end face may comprise a helix angle that is substantially identical to the helix angle of the spirally wound body.

The offset portion may at least partially receive the spirally wound body.

The offset portion may be at an end of the connector body.

The medical tube connector may comprise an offset portion at each end of the connector body.

The medical tube connector may comprise a guide configured to engage with the spirally wound body to guide the spirally wound body onto and/or along the offset portion. The guide may be positioned on the offset portion. The guide may comprise a channel configured to at least partially receive the spirally wound body. The guide may comprise a wall against which the spirally wound body abuts, when received in the channel. The guide may comprise a pair of opposed walls between which the spirally wound body is received. The channel may be a recess formed in the connector body.

The guide for the spirally wound member may comprise one or more engagement features configured to engage the spirally wound member, to assist in restraining and/or retaining the spirally wound member on the guide.

The engagement feature may comprise a protrusion that projects into the guide, the protrusion engaging the spirally wound member.

The engagement feature may comprise a plurality of protrusions.

The protrusions may comprise a pair of opposed protrusions, a gap being defined between the opposed protrusions. The gap may be smaller than the resting diameter of the spirally wound member.

The or each protrusion may comprise a rounded end.

The or each protrusion may comprise an inclined upper surface.

The medical tube may comprise a tube end retainer configured to receive, locate and retain an end of the spirally wound body. The tube end retainer may be positioned on the offset portion. The tube end retainer may be spaced from the guide along the offset portion.

The medical tube connector may comprise an overmould that encapsulates some or all of the connector body.

The overmould may extend beyond the ends of the connector body.

The overmould may extend over the tube.

The overmould may pneumatically connect the tube to the connector body.

The end face may be at least partially spaced along the axis of the lumen from the end of the tube.

According to another aspect of this disclosure there is provided an intermediate connector for a breathing limb of a breathing circuit, the breathing limb comprising:
a first tube segment comprising a first lumen, and a first heater wire;
a second tube segment comprising a second lumen, and a second heater wire; wherein
the intermediate connector is configured to pneumatically connect the first and second lumens, and to electrically connect the first and second heater wires; wherein
the intermediate connector comprises:
a pair of electrical connectors each configured to electrically connect with a respective heater wire; and
an electrical bridge extending between, and electrically connecting, each electrical connector with a respective heater wire;
   wherein the first wire extends at least partially around and/or along the exterior of the intermediate connector in a first direction from the first tube segment to the first electrical connector when the intermediate connector is viewed from a first end of the intermediate connector along a longitudinal axis extending through the lumen of the intermediate connector, and the second wire extends at least partially around and/or along the exterior of the intermediate connector in a second, opposite direction, from the second tube segment to the second electrical connector, when the intermediate connector is viewed from a first end of the intermediate connector along a longitudinal axis extending through the lumen of the intermediate connector;
wherein the intermediate connector comprises a probe port body comprising a probe port.

The electrical bridge may comprise a permanent electrical connection between each electrical connector and a respective heater wire.

According to another aspect of this disclosure there is provided a connector for a breathing limb of a breathing circuit, the breathing limb comprising:
a tube comprising a lumen, and a heater wire;
wherein the connector comprises a connector lumen and an electrical connector, the connector being configured to pneumatically connect the tube lumen with the connector lumen, and to electrically connect the heater wire to the electrical connector;
wherein the connector comprises a wire guide configured to receive the heater wire prior to the connection between the heater wire and the electrical connector; the wire guide comprising an enclosed channel configured to receive, locate and enclose a portion of the heater wire, and a wire outlet aperture adjacent the enclosed channel, and through which an end of the heater wire extends, to enable the end of the heater wire to be electrically connected to the electrical connector.

The wire guide may be configured to also function as a tube retainer, to receive, locate and retain part of the tube, such as the end or an end portion of the tube. For example, the tube may comprise a spirally wound body, the wire retainer being configured to receive, locate and retain an end of the spirally wound body.

Alternatively, the connector may comprise the wire guide, and a separate tube retainer. The wire guide and the tube retainer may be spaced apart around and/or along the connector.

The wire guide comprises a longitudinal axis, the or each channel extending in a direction generally parallel with the longitudinal axis.

The tube may comprise a plurality of heating wires, the connector comprising a plurality of enclosed channels, each configured to receive a respective wire.

The plurality of channels may be spaced apart.

The plurality of channels may be laterally spaced apart relative to the longitudinal axis. At least one channel may be laterally spaced from the longitudinal axis.

The wire guide may comprise a housing, the wire being received in an internal cavity of the housing.

The wire outlet aperture may be provided in an end wall of the housing.

The or each channel may extend through the end wall of the housing.

The or each channel may extend into the cavity of the housing.

The cavity may comprise an internal wall configured to at least partially define one or each channel.

The internal wall may be inclined with respect to the longitudinal axis of the wire retainer. The internal wall may therefore function as a wire deflector during manufacture, to deflect the heating wire toward an outlet aperture during manufacture.

The internal wall may be integral with the housing.

The end portions of the first and second heater wires may be overmoulded with the wire guide. The overmould material may flow into the housing cavity during manufacture.

The housing of the wire guide may comprise overmoulding features that facilitate the flow of overmould material into the housing cavity and around the heater wires.

The overmoulding feature may comprise an overmould indent at the base of the housing. The overmould indent may, when viewed from above, be between the wire outlet apertures. The overmould indent may be recessed below the base of the base of the housing of the wire guide.

The overmoulding feature may comprise an enlarged corner of the cavity of the wire guide, the corner being flared outwardly from the centre of the cavity when viewed from above so as to each define an enlarged space to receive overmould material during manufacture.

According to an aspect of this disclosure there is provided an inspiratory limb configured to transport breathable gases to a patient, the inspiratory limb comprising the intermediate connector or the tube connector of any one or more of the above statements.

According to an aspect of this disclosure there is provided an expiratory limb configured to transport breathable gases from a patient, the expiratory limb comprising the intermediate connector or the tube connector of any one or more of the above statements.

According to another aspect of this disclosure there is provided a respiratory therapy system comprising:
a) a humidifier;
b) a controller; and
c) the medical tube, and/or the intermediate connector and/or the tube connector of any one of the above statements.

The controller may be configured to control the system to:
deliver breathable gases to an upstream end of the first tube segment at a first temperature, for example a temperature of around 37° C.; and
increase the temperature of the breathable gases by a first predetermined amount to a second temperature, at the intermediate connector, for example to around 40° C.

The controller may be configured to provide heating control, of the first tube segment, using closed loop control.

The system may be configured to:
increase the temperature of the breathable gases by a second predetermined amount to a third temperature, for example to less than 42° C., in the second tube segment; and/or
   maintain the temperature of the breathable gases, from the intermediate connector to a patient end connector of the second tube segment substantially at the temperature of a temperature controlled environment (for example an incubator), for example 37° C.; and/or
prevent a decrease in temperature below 33° C.

The system may be configured to prevent a decrease in temperature below 31° C.

The heating control of the second tube segment may be via open loop control.

### BRIEF DESCRIPTION OF THE DRAWINGS

Throughout the drawings, reference numbers can be reused to indicate general correspondence between reference elements. The drawings are provided to illustrate example embodiments described herein and are not intended to limit the scope of the disclosure.

FIG. 1 is a schematic view of a respiratory humidification system comprising a breathing circuit, wherein the breathing circuit comprises one or more medical tubes and one or more tube end connectors in accordance with aspects of this disclosure.

FIG. 2 is an enlarged sectional view taken through the longitudinal axis of a medical tube in accordance with this disclosure.

FIG. 3 are side views of various medical tubes that can form part of a breathing circuit. These medical tubes include tubes that can comprise the inspiratory limb of the breathing circuit and include a dryline tube for transporting breathable gases between a gases source and a humidifier of a respiratory humidification system, and an inspiratory tube for transporting breathable gases from the humidifier to the patient. These medical tubes include tubes that can comprise the expiratory limb of the breathing circuit and include a relatively long expiratory tube for transporting expiratory gases from the patient to a water trap, and a relatively short expiratory tube for transporting expiratory gases from the water trap to the gases source.

FIGS. 4 to 6 are perspective views of example prior art respiratory humidification systems for delivering humidified gas to a user, the respiratory humidification system having a breathing circuit that optionally includes a non-heated inspiratory conduit. with FIG. 4 showing use of such a system with an infant warmer, but where the non-heated inspiratory conduit is not used, FIG. 5 showing use of such a system where the non-heated inspiratory conduit is located in an incubator, and FIG. 6 showing use of such a system where the patient is being held, where the non-heated inspiratory tube is not used.

FIG. 7 is a schematic view of an example prior art respiratory humidification system for delivering humidified gas to a user, the respiratory humidification system having a breathing circuit that includes a segmented inspiratory limb, with one segment being located in a temperature controlled environment such as an incubator.

FIG. 8 is a schematic view of respiratory humidification system for delivering humidified gas to a user in accordance with aspects of this disclosure, the respiratory humidification system having a breathing circuit that includes a segmented inspiratory limb, with one segment being located in a temperature controlled environment such as an incubator.

FIGS. 9 to 11 are perspective, side and top views of the segmented inspiratory limb of the system of FIG. 8.

FIG. 12 is a perspective view of an intermediate connector of the inspiratory limb of FIGS. 9 to 11.

FIG. 13 is a sectional side view of the intermediate connector of FIG. 12.

FIGS. 14 to 18 are respectively upper perspective, lower perspective, top, side, and bottom views of the intermediate connector of FIGS. 12 and 13, with one of the tubes omitted, and the overmould material omitted.

FIG. 19 is a perspective view of a tube end connector, connected to one of the tubes of the inspiratory conduit, the tube end connector being a patient end connector.

FIGS. 20 and 21 are perspective and side views of a device end connector of the system of FIG. 8, connected to one of the tubes of the inspiratory conduit, and FIGS. 22) to 26 are respectively perspective, top, side, bottom and end views of the device end connector, with the overmould material omitted.

FIGS. 27 to 30 are side views of respectively the intermediate connector of FIG. 12, the patient end connector of FIG. 19, and the device end connector of FIGS. 20 to 26, with FIG. 30 being an enlarged view of the cutaway portion of FIG. 28. Each of these views shows a transparent portion, so that the wires can be more clearly seen.

FIGS. 31 and 32 are respectively enlarged perspective and side views of a tube end connector and medical tube segment in accordance with this disclosure, being a patient end connector that could be used with/comprise part of the inspiratory tube of FIG. 3.

FIG. 33 is a sectional view of the tube end connector and medical tube segment of FIGS. 31 and 32.

FIG. 34 is a perspective view of the tube end connector and medical tube segment of FIGS. 31 and 32, with the overmould material of the tube end connector removed to more clearly show a terminal retainer of the tube end connector.

FIG. 35 is an enlarged side view corresponding to FIG. 34.

FIG. 36 is a sectioned view along the longitudinal axis of a lumen of the tube end connector of FIGS. 34 and 35.

FIG. 37 is an enlarged view of the terminal retainer of FIGS. 34 and 35 in a direction perpendicular to the longitudinal axis of the lumen of the tube.

FIG. 38 is an enlarged view of the terminal retainer of FIGS. 34 and 35 in a direction along the longitudinal axis of the lumen of the tube.

FIGS. 39 to 44 are respectively perspective, side, part sectional end, top, end, and sectional side views of a tube end connector in accordance with this disclosure.

FIG. 45 is a perspective view of a tube end connector in accordance with this disclosure.

FIG. 46 is a perspective view of a tube end connector in accordance with this disclosure.

FIGS. 47 to 50 are respectively perspective, side, end, and top views of a tube end connector in accordance with this disclosure.

FIG. 51 is a circuit diagram showing the electrical connections between the first and second tube segments and the electrical power output from a system controller.

FIG. 52 is a perspective view from underneath of another intermediate connector of the inspiratory limb of FIGS. 9-11, in accordance with this disclosure.

FIG. 53 is a perspective view from above of the intermediate connector of FIG. 52.

FIG. 54 is a side view of the intermediate connector of FIG. 52.

FIG. 55 is a plan view of the intermediate connector of FIG. 52.

FIG. 56 is a view from underneath of the intermediate connector of FIG. 52.

FIG. 57 is an end view of the intermediate connector of FIG. 52.

FIG. 58 is a part sectional side view of the intermediate connector of FIG. 52.

FIG. 59 is a sectional side view of the intermediate connector of FIG. 52, taken through the longitudinal axis of the gases flow lumen of the intermediate connector.

FIG. 60 is a sectional end view of the intermediate connector of FIG. 52.

FIG. 61 is a perspective view from underneath of the intermediate connector of FIG. 52.

FIG. 62 is a perspective view from above of the intermediate connector of FIG. 52.

FIG. 63 is an enlarged view from above of a wire guide of the intermediate connector of FIG. 52.

FIG. 64 is an enlarged, sectional side view of an embodiment of the wire guide of FIG. 63.

FIG. 65 is an enlarged, sectional side view of another embodiment of the wire guide of FIG. 63.

FIG. 66 is an enlarged top view of a bead guide of the intermediate connector of FIG. 52.

FIG. 67 is an enlarged side view of the bead guide of FIG. 66.

FIG. 68 is an enlarged, sectional side view of the bead guide of FIG. 66.

FIG. 69 is an exploded perspective view from above and one end of a device end connector in accordance with this disclosure, for use with the system of FIG. 8 with an electrical socket connector mount not mounted on the device end connector, and an electrical connector socket omitted.

FIGS. 70 and 71 are perspective views from the top of the device end connector of FIG. 69, with the electrical socket connector mount mounted to the device end connector, and the electrical connector socket mounted on the electrical socket connector mount.

FIGS. 72) to 74 are respectively top, end and side views corresponding to FIGS. 70 and 71.

FIG. 75 is a perspective view of the device end connector of FIGS. 70 and 71 with the electrical socket connector mount mounted to the device end connector, and with the electrical connector socket removed.

FIG. 76 is an enlarged perspective exploded view of the device end connector of FIGS. 70 and 71 with the electrical socket connector mount mounted to the device end connector, also showing the tube.

FIG. 77 is a further enlarged exploded view corresponding to FIG. 76.

FIG. 78 is a part sectional, enlarged perspective view from the top, showing the device end connector of FIGS. 70 and 71 connected to a tube.

FIG. 79 is a sectional top view of the device end connector of FIGS. 70 and 71.

### DETAILED DESCRIPTION

Embodiments of medical tubes, tube end connectors, components and methods of assembly and manufacture will now be described with reference to the accompanying figures, wherein like numerals refer to like or similar elements throughout. Although several embodiments, examples and illustrations are disclosed below, it will be understood by those of ordinary skill in the art that the disclosures described herein extend beyond the specifically disclosed embodiments, examples and illustrations, and can include other uses of the disclosures and obvious modifications and equivalents thereof. The terminology used in the description presented herein is not intended to be interpreted in any limited or restrictive manner simply because it is being used in conjunction with a detailed description of certain specific embodiments of the disclosures. In addition, embodiments of the disclosures can comprise several novel features and no single feature is solely responsible for its desirable attributes or is essential to practicing the disclosures herein described.

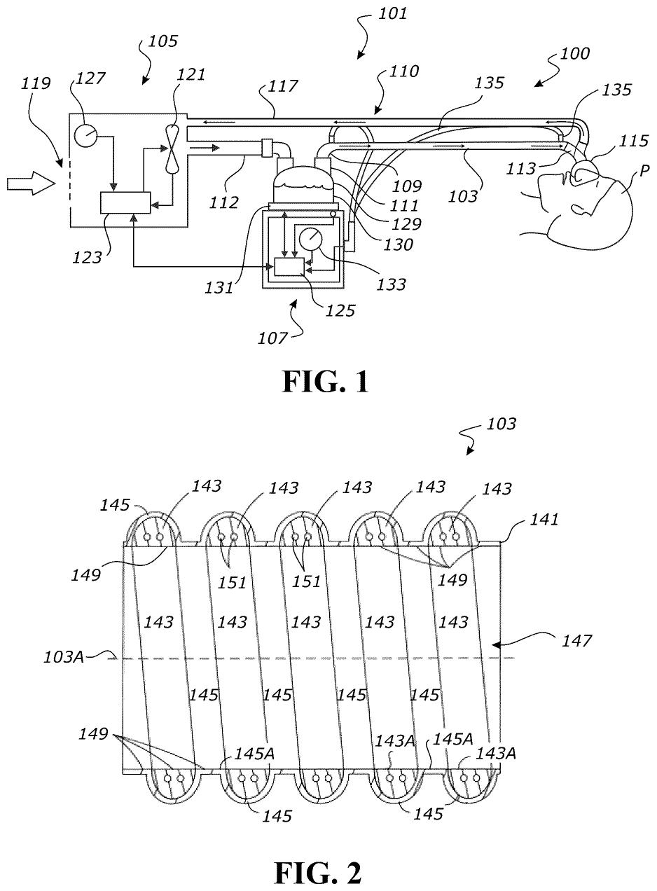

For a more detailed understanding of the disclosure, reference is first made to FIG. 1, which shows a breathing circuit 100 comprising part of a respiratory humidification system 101. Such a breathing circuit 100 can be a respiratory humidification circuit. The breathing circuit 100 includes one or more medical tubes. The breathing circuit 100 can include an inspiratory limb comprising an inspiratory tube 103 and an expiratory limb 110 comprising an expiratory tube 117. The ends of the medical tubes each comprise, or are provided with, a tube end connector.

As used herein, medical tube is a broad term and is to be given its ordinary and customary meaning to a person of ordinary skill in the art (that is, it is not to be limited to a special or customized meaning) and includes, without limitation, cylindrical and non-cylindrical elongate shapes defining a lumen or comprising a passageway, such as a hollow, elongate body that are configured for use in medical procedures and that otherwise meet applicable standards for such uses. An inspiratory tube is a medical tube that is configured to deliver breathing gases to a patient. An expiratory tube is a medical tube that is configured to remove exhaled gases away from a patient.

Gases can be transported in the circuit 100 of FIG. 1. Breathable gases, such as ambient gases and/or oxygen flow from a gases source 105 to a humidifier 107 via a dryline tube 112, which is typically unheated. The humidifier 107 can humidify the gases. The gases source 105 can be a ventilator, a blower or fan, a tank containing compressed gases, a wall supply in a medical facility, any other suitable source of breathing gases, or any combination. The gases source 105 and humidifier 107 may be integrated into a common base unit (not shown in this example) comprising a common housing that houses the blower or fan, and on which the humidifier 107 is mounted. The humidifier 107 may comprise a water chamber 129, the water chamber 129 being removably mounted on the base unit.

The humidifier 107 connects to an inlet 109 (the end for receiving humidified gases) of the inspiratory tube 103 via a port 111, thereby supplying humidified gases to the inspiratory tube 103. The gases flow through the inspiratory tube 103 to an outlet 113 (the end for expelling humidified gases) of the inspiratory tube 103, and then to a patient P through a patient interface 115 connected to the outlet 113. The expiratory tube 117 connects to the patient interface 115. The expiratory tube 117 returns exhaled humidified gases from the patient interface 115 to the gases source 105 or to the ambient atmosphere.

As used herein, patient interface has a broad meaning and is to be given its ordinary and customary meaning to one of skill in the art, and patient interface also includes, without any limitation, any one or more of a full face mask, a nasal mask, an oral mask, an oral-nasal mask, a nasal pillows mask, nasal cannula, nasal prong(s), a laryngeal mask, or any other suitable coupling between the breathing circuit and the airways of the patient. Such a patient interface or coupling could comprise a tracheal tube for example. The breathing circuit may comprise a wye-piece, which may be integral with, comprise part of, or be configured to be connected to the patient interface. The wye-piece comprises three ports. One port connects to or is integral with the patient interface, one port of the wye-piece is configured to be connected to the inspiratory limb of the breathing circuit, and the other port is configured to be connected to the expiratory limb of the breathing circuit.

Gases can enter the gases source 105 through a vent 119. The blower or the fan 121 can cause gases to flow into the gases source 105 by drawing air or other gases through the vent 119. The blower or the fan 121 can be a variable speed blower or fan. An electronic controller 123 can control the blower or fan speed. In particular, the function of the electronic controller 123 can be controlled by an electronic master controller 125. The function can be controlled in response to inputs from the master controller 125 and/or a user-set predetermined required value (preset value) of pressure or blower or fan speed input via controller 125, or via a dial or other suitable input device 127.

The humidifier 107 comprises a humidification chamber 129. The humidifier chamber 129 can be configured to contain a volume of water 130 or other suitable humidifying liquid. The humidification chamber 129 can be removable from the humidifier 107. Removability allows the humidification chamber 129 to be more readily sterilized or disposed of after use. The humidification chamber 129 portion of the humidifier 107 can be a unitary construction or can be formed of multiple components that are joined together to define the humidifier chamber 129. The body of the humidification chamber 129 can be formed from a non-conductive glass or plastics material. The humidification chamber 129 can also include conductive components. For instance, the humidification chamber 129 can include a highly heat conductive base (for example an aluminium base) configured to contact or be associated with a heater plate 131 on the humidifier 107 when the humidification chamber 129 is installed on the humidifier 107.

The humidifier 107 can include electronic controls. The humidifier 107 can include the electronic, analog or digital master controller 125. The master controller 125 can be a microprocessor-based controller executing computer software commands stored in associated memory. In response to the user-set humidity or temperature value input via a user input device 133 and other inputs, the master controller 125 determines when (or to what level) to energize the heater plate 131 to heat the volume of water 130 within the humidification chamber 129.

A temperature probe 135 can connect to the inspiratory tube 103 near the patient interface 115 or the temperature probe 135 can connect to the patient interface 115. The temperature probe may extend into the inspiratory tube 103 via a probe port formed in a probe port body at or near the end of the tube 103, for example at a tube end connector. The temperature probe 135 can be integrated into the inspiratory tube 103, and is typically associated with a sensor wire that may extend along the inspiratory tube. The temperature probe 135 detects the temperature near or at the patient interface 115. A signal reflecting the temperature can be provided by the temperature probe 135 to the electronic, analog or digital master controller 125. A heating element (not shown), but typically one or more heater wires extending along the inspiratory tube, can be used to adjust the temperature of the patient interface 115 and/or the inspiratory tube 103 to raise the temperature of the inspiratory tube 103 and/or the patient interface 115 above the saturation temperature, thereby reducing the opportunity for unwanted condensation.

In the respiratory humidification system 101 of FIG. 1, exhaled humidified gases are returned from the patient interface 115 to the gases source 105 via the expiratory tube 117. The expiratory tube 117 can include a vapor permeable material. The vapor permeable expiratory tube can be corrugated.

The expiratory tube 117 may be configured to be connected to a water trap, configured to receive condensate that is present, or which forms, in the expiratory tube 117.

The expiratory tube 117 can have a temperature probe and/or heating element, as described above with respect to the inspiratory tube 103, to reduce the opportunity for condensation to reach the gases source 105. The expiratory tube 117 need not return exhaled gases to the gases source 105. The exhaled humidified gases can flow directly to ambient surroundings or to other ancillary equipment, such as an air scrubber/filter (not shown).

FIG. 2 is a cross-sectional view of a portion of a medical tube being, for example, an inspiratory tube 103 as described above, taken through the longitudinal axis 103A of the tube 103. As illustrated, the inspiratory tube 103 comprises a tube body 141 which can be formed from a bead 143 and tape or film 145. As will be described in greater detail, the bead 143 and the film 145 can define a bore (or inner lumen) 147 of the medical tube 103. An inner surface 149 of the medical tube 103 may at least partially define the bore 147. The inner surface 149 can be substantially smooth. Thus, the bore 147 can be considered a substantially smooth bore.

As will be described in greater detail below, the inner surface 149 can comprise alternating portions of the bead 143 and the tape or film 145. For example, as illustrated in FIG. 2, the inner surface 149 can comprise portions 143A of the bead 143 and portions 145A of the film 145. The portions 143A of the bead 143 and the portions 145A of the film 145 can be substantially flat as described below. It should be appreciated by one of skill in the art that the bead 143, which may be much thicker and/or made of a harder or more rigid material than the film 145, may impart structural support, reinforcement, and/or resistance to crushing to the more flexible film portions of the tube 103. Such structural support, reinforcement, and/or resistance to crushing may be important in medical tubes, and in particular for breathing tubes, which must meet international standards defining usage characteristics and parameters.

As shown in FIG. 2, in some embodiments various components can be embedded within the bead 143. For example, in the illustrated embodiment, a heating element comprising optionally two heater wires 151 is disposed within the bead 143. In some embodiments, the two heater wires 151 may comprises a single wire that extends from one end of the tube 103 to the other end of the tube 103 and then is doubled back such that both ends of the heater wire 151 can be attached to a single connection point on one end of the tube. The other end of the tube 103 may therefore comprise an end portion of the wire 151 being the portion of the wire 151 that is looped back on itself to return to the first end of the tube 103. Where the heating element comprises two heater wires 151, the ends of these may be twisted, soldered, or otherwise connected together to form the end portion of the wire 151.

In some embodiments, the heating element may comprise a single heater wire, or greater than two heater wires.

In some embodiments, other components, such as one or more sensing and/or ground or earth wires may also be disposed within the bead 143. Sensor wire(s) embedded within the bead 143 can be configured to measure, for example, temperature, pressure, flow, or humidity. Such heating and/or sensing and/or ground or earth wires 151 may be any suitable size considering the overall bead size. In one embodiment, the wires 151 are in the range of 0.05-1.0 mm, 0.1-0.8 mm, 0.1-0.4 mm. Overall, there may be one, two, three, four or more wires 151 within the bead 143, for heating and/or sensing and/or ground or earth functions. In another embodiment, the wires 151 are wound in a pattern or laid through the lumen of the tube, rather than being embedded or encapsulated in the bead. In such an embodiment, the wires 151 may be held in place at one or both ends.

Because the bead 143 forms a portion of the inner surface 149 that defines the bore, the heating element 151 is positioned in close proximity to gases within the medical tube 103. Positioning one or more heating elements 151 within the bead 143 of the tube 103 can maximize humidification, minimize condensate formation, maintain the condition of the gases, and contribute to the efficiency of the tube 103 and/or any associated breathing circuit or the humidification system.

Advantageously, locating the wires in the bead 143 (rather than in the lumen 147) reduces the resistance to flow of the tube 103. In some embodiments, the heating elements 151 (and/or other sensor and/or ground or earth wires) can be similarly embedded or positioned within the film 145. It should further be appreciated by one of skill in the art that the benefit of two relatively small wires 151 (relative to the size of the bead 143) located in the portion of the bead 143 close to the inner surface of the tube wall is that this can be an efficient way to heat the gas flowing through the tube 103, while reducing the risk of unacceptably high temperatures on the outer surface of the tube.

International standards for breathing circuits govern the maximum outer surface temperature to limit the risk of burning a patient or carer. Accordingly, in some embodiments, the primary purpose of such heater wires 151 is not for reinforcement, but for maintaining water in (or re-evaporating water to) a vapor state in the tube 103 and reducing the risk that such vapor condenses into (or stays condensed as) liquid water and causes damage to the patient or other equipment. Moreover, in some embodiments a pair or pairs of wires 151 are preferable (as compared to a single wire), especially in a medical tube as described herein, to provide for a length of wire doubled back along the length of the tube to a single connection point (usually at one end of the tube) to a power supply/ground. The single connection point offers costs savings, reliability, functionality, and usability benefits over other design.

The medical tubes disclosed herein may be configured for a neonatal, paediatric, or adult patient population. Amongst other things, this inner diameter of the tube can be varied for different parts of the population. For example, the adult tube may have a larger internal diameter than a neonatal tube. For some applications, the neonatal tube may have a smaller internal diameter. A smaller internal diameter may reduce the compliance of the breathing circuit, thereby reducing the possibility for barotrauma in volume-controlled therapy.

The above description uses inspiratory tube 103 as an example of a medical tube. However, it will be appreciated that the medical tube could comprise any other tube, or multiple tube segments, of the breathing circuit 100.

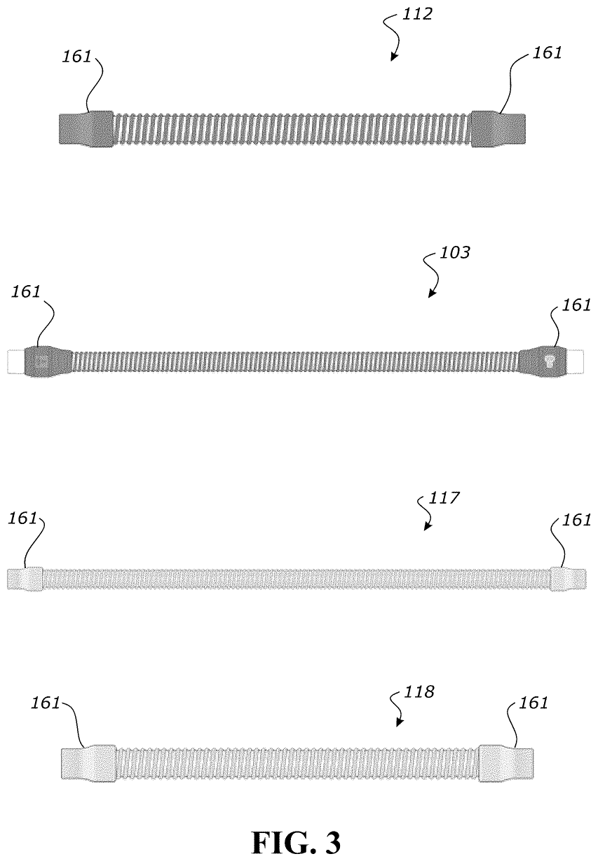

With reference to FIG. 3, examples of a medical tube, for use in a breathing circuit, such as breathing circuit 100, of a respiratory humidification system, are provided. FIG. 3 shows:
a) A dryline tube 112 configured to be connected between the gases source 105 and the humidifier 107.
b) An inspiratory tube 103 configured to be connected between the humidifier 107 and the patient interface 115.
c) A relatively long expiratory tube 117 configured to be connected between the patient interface 115 and a water trap.
d) A relatively short expiratory tube 118 configured to be connected between the water trap and the gases source 105.

Each end of each tube is provided with an end connector 161 to connect the end of the tube to another component of the respiratory therapy apparatus, for example to another tube, the patient interface 115, the water trap (not shown), a wye-piece 409, another tube connector, or to the humidifier 107, and/or one or more sensors. The end connectors 161 may be removably connected to the tube ends, or may be permanently connected to the tube ends. For example, a tube end may be positioned adjacent an end connector, and pneumatically connected using an overmould which extends over the tube end and over the connector.

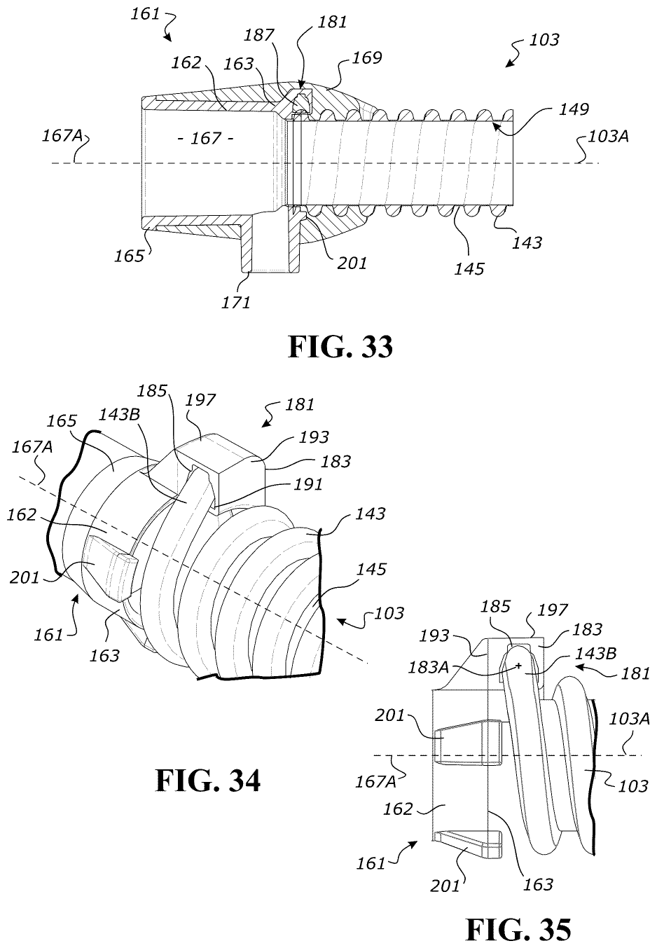

With reference for example to FIGS. 32 and 33 each end connector 161 comprises a substantially rigid cylindrical connector body 162 which comprises a first end 163 adjacent an end of the medical tube, and a second end 165 that connects to another component. The second end 165 may be female or male to connect with a corresponding male or female connector on the component.

Each connector body 162 comprises a central lumen 167 extending along and forming a gases flow path through the connector body 162 from the first end 163 to the second end 165, the lumen comprising a lumen axis 167A. With reference to FIG. 32, each end connector 161 comprises an outer cuff 169, that substantially covers the rigid connector body 162.

The outer cuff 169 may comprise a resiliently deformable material.

The outer cuff 169 may comprise overmould material that covers the rigid connector body 162 and the end of the tube. The overmould material 169 may provide the structural and sealing connection between the tube and the connector body 162.

The end of the tube may be secured to the first end 163 of the end connector body 162 for example by any one or more of: threading the end of the tube onto corresponding threads on the connector body 162; adhesive; and/or overmoulding. In the below described embodiments, it is preferred that the tube is not threaded onto the end connector body 162. It is preferred that the tube is connected to the end connector 161 only via overmould material.

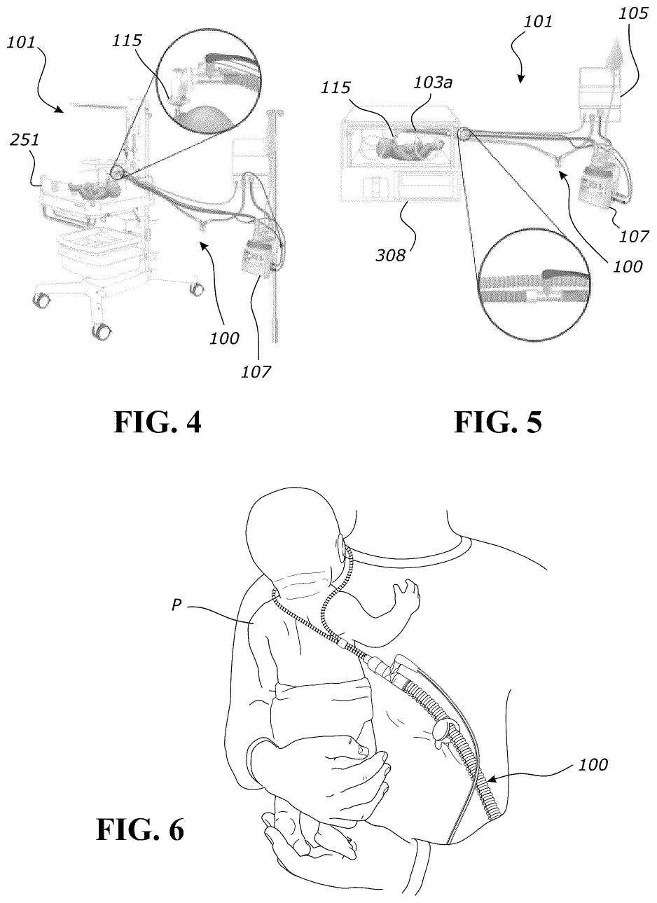

With reference to FIG. 4 onwards, when a medical tube comprising a heated, humidified breathing tube is used for an incubator 308 (or any region where there is a temperature change, such as around radiant warmers, or under a blanket used by a patient), or in any other temperature controlled environment, the breathing tube will pass through at least two distinct zones: a lower temperature zone (such as the one outside the incubator) and a higher temperature zone (such as the one inside the incubator). If the tube is heated along its full length, one of the zones may tend to be at an undesirable, unsuitable, or non-optimal temperature, depending on which zone temperature is sensed (e.g. which zone contains a temperature sensor). If a heater wire of the tube is controlled to a sensor inside the incubator 308 (such as to a patient-end temperature sensor), the section outside the incubator 308 may tend to be too cool, which can lead to condensation. Conversely, if the heater wire is controlled to a sensor outside the incubator 308, the section inside the incubator 308 may tend to be too hot, which can lead to overheated gas being provided to the patient P and/or to the external surface of the tube exceeding a safe level.

With reference to FIGS. 4 to 6, a prior art respiratory humidification system 101 having a breathing circuit 100 can optionally include a non-heated inspiratory conduit segment 103a. FIG. 4 shows use of such a system with an infant warmer 251, but where the non-heated inspiratory conduit segment 103a is not used. FIG. 5 shows use of such a system where the non-heated inspiratory conduit segment 103a is located in an incubator 308, and FIG. 6 shows use of such a system where the patient P is being held, where the non-heated inspiratory tube segment 103a is not used.

Breathing circuits ideally avoid or minimise “rainout” (the creation of condensate due to a drop in gases temperature), avoid or minimise overheated gases (delivering gases to the patient interface which may pose a risk to the patient), and avoid or minimise excessive surface temperature of the inspiratory limb (where the external surface temperature of the inspiratory limb may pose a risk to the patient). This can be particularly important for the portion of the inspiratory limb at the patient end of the circuit, for example the 300 mm of the inspiratory circuit that is nearest the patient. All of these considerations are even more important when providing a system for providing humidified breathable gases to a neonate, where the flow rate of breathable gases delivered are typically relatively low (for example 0.5-4 L/min).

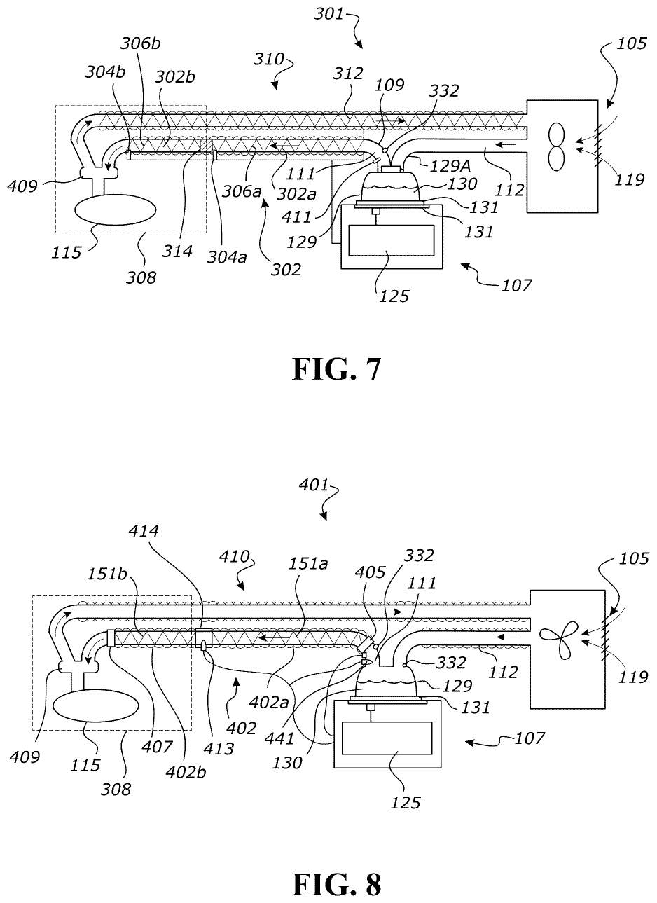

With reference to FIG. 7, which will be described in more detail below, a prior art respiratory humidification system 301 is shown comprising an inspiratory limb 302 comprising a medical tube comprising two segments of tube 302a, 302b, one inside and one outside the incubator 308. Each tube segment 302a, 302b comprises its own heater wire which can be independently controlled by the system controller 125. The controller 125 can therefore independently control and vary the heat output of the two tube segments 302a, 302b.

Prior art system 301 can provide independent control of the heating provided by the inspiratory limb 302 inside and outside of the incubator 308.

However, it can be desirable to be able to provide a breathing circuit, with a heated inspiratory and/or expiratory limb, where the heating provided inside and outside of the incubator 308 is different, but where a control algorithm which independently controls the electrical power delivered to each tube segment is not required.

In the prior art system 301 above, and the system 401 described below in accordance with this disclosure, the system includes an incubator 308. However, incubator 308 is just an example of a temperature controlled environment in which the heating required from a medical tube inside the temperature controlled environment is different to the heating required outside of the temperature controlled environment.

With reference to FIG. 7 an example prior art respiratory humidification system 301 for delivering humidified gas to a user has many similar components to that of system 101 described above with reference to FIG. 1.

The respiratory humidification system 301 comprises a breathing circuit 300 that includes a segmented inspiratory limb 302 with sensors 304a, 304b in each segment. The segmented inspiratory limb 302 can be used in conjunction with an incubator 308, as illustrated, or with another system where there are different temperatures along different segments of the inspiratory limb 302, such as in conjunction with a radiant warmer, or in any other temperature controlled environment. The segmented inspiratory limb 302 can be used to provide different levels of heat to different segments of the inspiratory limb 302a, 302b to reduce or prevent condensation and/or to control a temperature of gas delivered to a user.

While other types of humidification units can be used with certain features, aspects, and advantages described in the present disclosure, the illustrated humidification unit 107 is a pass-over humidifier that comprises a humidification chamber 129 adapted to contact a heater plate 131. The heater plate 131 can be controlled through a controller 125 or other suitable component such that the heat transferred into the liquid 130 in the humidification chamber 129 can be varied and controlled.

The inspiratory limb 302 is connected to outlet 111 of the humidification unit 107. The inspiratory limb 302 conveys toward a user the mixture of gases and water vapor that exits the humidification chamber 129. The inspiratory limb 302 can include a heating element 306a, 306b positioned along the inspiratory limb 302, wherein the heating elements 306a, 306b are configured to reduce condensation along the inspiratory limb 302, to control a temperature of gas arriving at the user, to maintain humidity of the gas, or any combination of these. The heating elements 306a, 306b can raise, maintain the temperature of, or minimize a temperature drop of, the gases and water vapor mixture being conveyed by the inspiratory limb 302. In some implementations, the heating elements 306a, 306b can be a wire, such as a wire 151 as described above, that defines a resistance heater. By increasing or maintaining the temperature of the gases and water vapor mixture leaving the humidification chamber 129, the water vapor is less likely to condensate out of the mixture.

The respiratory humidification system 301 can be used in conjunction with an incubator 308. The incubator 308 can be configured to maintain a desired environment for a user within the incubator 308, such as a selected, defined, or desired temperature. Within the incubator 308, therefore, an interior temperature may be different than a temperature outside the incubator 308. Thus, the incubator 308 causes, defines, creates, or maintains different temperature zones along the inspiratory limb 302, where the interior temperature is typically hotter than the exterior temperature.

The respiratory humidification system 301 can include an expiratory limb 310, which can include an associated heating element 312. In some embodiments, the expiratory limb 310 and the inspiratory limb 302 can be connected using a suitable fitting (e.g., a wye-piece 409).

The inspiratory limb 302 can be divided into segments 302a and 302b where a first segment 302a can be a portion of the inspiratory limb 302 that is outside the incubator 308 and a second segment 302b (e.g., an incubator extension), can be a portion of the inspiratory limb 302 that is inside the incubator 308.

The respiratory humidification system 301 can include an intermediate connector 314 that can be configured to pneumatically and electrically couple elements of the first and second segments 302a, 302b of the inspiratory limb 302. The intermediate connector 314 can be configured to electrically connect the heater wires 306a in the first segment 302a to the heater wires 306b in the second segment 302b to enable control of the heater wires 306a, 306b using the controller 125.

The inspiratory limb 302 can include sensors 304a, 304b in respective segments of the inspiratory limb 302a, 302b. The first sensor 304a can be positioned near an end of the first segment 302a, close to the incubator 308 so that the parameter derived from the first sensor 304a corresponds to a parameter of the humidified gas entering the second segment 302b. The second sensor 304b can be positioned near an end of the second segment 302b so that the parameter derived from the second sensor 304b corresponds to a parameter of the humidified gas delivered to the patient or user. The output of the sensors 304a, 304b can be sent to the controller 125 as feedback for use in controlling power delivered to the heating elements 306a, 306b of the segments of the inspiratory limb 302a, 302b. In some embodiments, one or both of the sensors 304a, 304b can be temperature sensors, humidity sensors, oxygen sensors, flow sensors, or the like. System 301 may further comprise a temperature sensor 411 at the outlet of the humidification chamber 129.

The controller 125 in prior art system 301 can be configured to control the heater wires 306a and 306b, to receive feedback from the sensors 304a and 304b, to provide logic to control power independently to the heater wires 306a and 306b, and to adjust control of the heater wires 306a and 306b in response to readings from the sensors 304a and 304b.

The controller 125 in prior art system 301 can independently control the amount of power delivered to the first and second segments 302a, 302b of the inspiratory limb 302.

Aspects of the current disclosure are configured to provide a medical tube comprising multiple tube segments that are configured to provide different levels of heating.

Aspects of the current disclosure are configured to provide a medical tube comprising multiple tube segments each having a respective tube heater, where the tube heaters are controlled by a single controller, and whereby only one tube segment is directly electrically connected to the controller. A second tube segment may be electrically connected only to the first tube segment. In embodiments, a downstream tube segment may only receive electrical power via the upstream tube segment, such that the downstream tube segment is a secondary tube segment, the upstream tube segment being a primary tube segment.

The tube segments may be always powered at the same time. The tube segments may be electrically connected in series. In use, the electrical current in both tube segments may always be the same.

In some embodiments in accordance with this disclosure, the ratio of the power delivered to the first tube segment and to the second tube segment can be configured by one or more characteristics of one or both tube segments. For example, the ratio of power delivered to each tube segment can be configured such that each tube segment is heated to a temperature to reduce or eliminate condensation. As a further example, the ratio of power can be selected so that overheated gas is not provided to the patient.

Embodiments provide for a medical tube comprising heater wires that are not within the gas path, that is, not within the tube lumen, but are contained within a material that separates them from the gas path and that also insulates them from an external environment. For example, the heater wires may be encapsulated in a bead of the tube. In some embodiments, the heater wire is molded into the inspiratory or expiratory tube such that the ends of the heater wires in complementary segments of the tube contact an intermediate connector such that the heater wires electrically couple to the intermediate connector.

Some embodiments described herein provide for a respiratory humidification system that is configured to deliver warm, humidified gas to a patient or other user. The gas is passed through a liquid chamber which is filled with a liquid (e.g., water) that is heated using a heater plate. The liquid evaporates in the chamber and combines with the gas which flows over it, thereby heating and/or humidifying the gas. The humidified gas can be directed to an inspiratory limb having one or more heater wires associated therewith. The heater wires can be powered to provide a defined, desired, appropriate, or selected amount of heat to the humidified gas.

In some embodiments, the respiratory humidification system can be used in conjunction with an incubator or radiant warmer. The inspiratory limb can be segmented such that a first segment is outside the incubator and a second segment is inside the incubator. Furthermore, a first set of heater wires can be associated with the first segment and a second set of heater wires can be associated with the second segment.

In some embodiments, the humidification system can include an expiratory limb having associated heater wires which are also controlled by the humidification system.

In this application, the segmented medical tube is described with reference to an inspiratory limb. However, the described features can be applied alternatively or additionally to an expiratory limb.

We refer now to FIG. 8 which shows a respiratory humidification system 401 in accordance with this disclosure. System 401 has many features common with the systems 101 and 301 described above.

System 401 comprises an inspiratory limb 402 comprising a medical tube comprising first and second tube segments 402a, 402b, first segment 402a being located outside of the incubator 308, second segment 402b being located inside the incubator 308. The two segments 402a, 402b are pneumatically and electrically connected together using an intermediate connector 414.

First segment 402a comprises a device end connector 405 configured to be connected to the humidifier outlet 111. The opposed, downstream end of first segment 402a connects to the intermediate connector 414. The upstream end of second segment 402b also connects to the intermediate connector 414, and therefore is pneumatically connected to the first segment 402a. The downstream end of second segment 402b comprises a patient end connector 407 which may connect to a wye-piece 409. The wye-piece 409 comprises three ports: one port is connected to patient end connector 407, another to the patient interface 115, and the third to expiratory limb 410.

Each segment 402a, 402b of the inspiratory limb 402 comprises a respective heater wire 151A, 151i, or in particular two heater wires in each segment 402a, 402b which together provide an electrical heating circuit configured to heat both segments 402a, 402b.

Two temperature sensors are provided in this embodiment. A first sensor 411 is provided at the humidifier outlet 111, for example on the outlet 111 itself, or on the device end connector 405. A second sensor 413 is provided at the intermediate connector 414, but external of the incubator 308. According to this disclosure the temperature of gases in the inspiratory conduit 402 inside the incubator 308 is not measured. The temperature is only measured external of the incubator 308, and in this example at the upstream and downstream ends of the first segment 402a of the inspiratory conduit 402. Optionally a flow sensor 332 can be provided, for example at: the inlet of the humidifier 129, at the outlet of the humidifier 129 (as can be seen with reference to FIG. 7), or on the inspiratory tube end connector that connects to the outlet of the humidifier 129. The flow sensor 332 could be located in any other desired position on the breathing circuit, the humidifier, or the flow generator. More than one flow sensor 332 could be provided.

In this embodiment both sensors 411, 413 are connected via flying leads to controller 125. The device end connector 405 is also electrically connected to the controller 125 via a further flying lead, so that the controller can provide electrical power to the device end connector 405 and therefore to the heater wires 151A, 151B.

The device end connector 405 and the patient end connector 407 can comprise a rigid internal connector body over which is overmould material that connects the connector body to the tube segments. The rigid internal connector body may comprise an assembly of a plurality of rigid connector components.

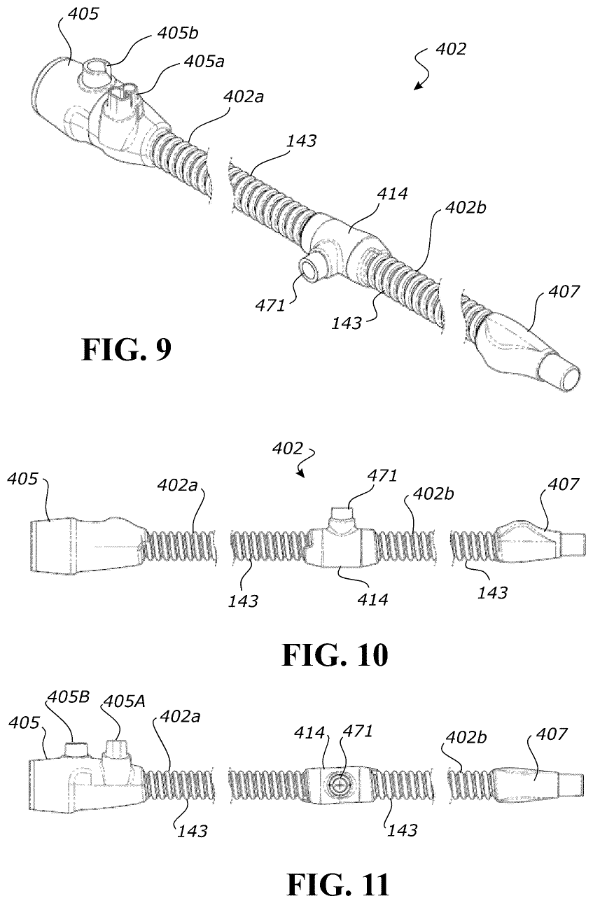

With additional reference to FIGS. 9-11, the inspiratory limb 402 can be seen comprising end connectors 405, 407 comprising rigid internal connector bodies which are overmoulded to provide the pneumatically sealed connection to the tube segments 402a, 402b. The intermediate connector 414 is similarly constructed, and will be described in greater detail below. The device end connector 405 comprises an electrical connector socket 405A for electrical connection via flying lead to the controller 125. Device end connector 405 also comprises a device end probe port 450B for receiving and locating device end sensor 411. Intermediate connector 414 is provided with a probe port 471 for the intermediate connector sensor 413. The probe port 471 is formed in a probe port body. Overmould material may extend around the probe port body, but not over the probe port itself.

The total length of the inspiratory limb from the distal end of the device end connector 405 to the distal end of the patient end connector 407 may be between 1.5 and 1.6 m. The distance between the distal end of the device end connector 405 and the midpoint of the intermediate connector 414 may be between 1.2 and 1.3 m.

The first segment 402A may be between 1100 and 1300 mm long in one example, from the distal end of device end connector 405 to the centre of intermediate connector probe port 471. The first segment 402A may be between 1200 and 1300 mm long.

The second segment 402b may be shorter, and in one example is around 200-400 mm long, from the centre of intermediate connector probe port 471 to the distal end of patient end connector 407. The second segment may be about 350 mm long.

The segmented inspiratory limb 402 comprises the medical tube comprising the first segment 402a and the second segment 402b, and the intermediate connector 414 configured to connect the first heater wires 151a to the second heater wires 151b. Connecting the two segments 402a and 402b can comprise pneumatically connecting the segments 402a, 402b to form a single conduit through which humidified gases can be delivered to a user, and electrically connecting the respective heater wires 151a, 151b through the intermediate connector 414.

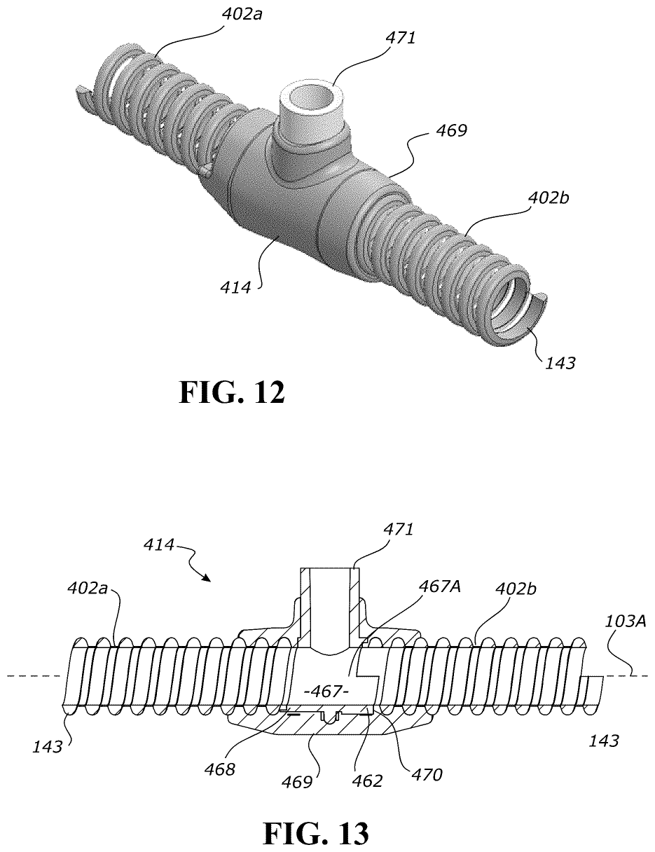

With reference to FIGS. 12 to 18, the intermediate connector 414 will be described in more detail.

The intermediate connector 414 comprises an internal rigid connector body 462 defining a gas flow lumen 467 comprising a lumen axis 467A. The lumen axis 467A extends through and between opposed ends 468, 470 of the intermediate connector body 462.

The rigid connector body 462 further comprises a hollow elongate probe port 471 which projects orthogonally away from the lumen axis 467A, to receive a sensor probe (not shown). The end of first inspiratory tube segment 402A abuts one end 468 of the rigid connector body 462. An end of second inspiratory tube segment 402B abuts the other end 470 of the rigid connector body 462.

The ends of each inspiratory tube segment 402a, 402b, the ends 468, 470 of the connector body 462, and the remainder of the connector body 462 are overmoulded 469 to mechanically and pneumatically connect the tube segments 402a, 402b to the intermediate connector 414.

The probe port 471 is also overmoulded, although the distal end of the probe port 471 is not overmoulded in this example, and projects from the overmould 469. The tube ends are spaced from the respective ends 468, 470 of the connector body 462 such that the overmould material fills the spaces between the tube ends and the connector body ends 468, 470. The overmould material in these spaces forms part of the lumen of the connector 414, so that there is a smooth transition between the tubes, overmould and the rigid connector body 462.

The probe port 471 may be at a midpoint of the intermediate connector 414.

The probe port 471 is intermediate the two heated inspiratory tube segments 402a, 402b.

The outer diameter of the probe port body may be 12 mm. The distal end of the probe port body may extend 22 mm from the lumen axis 103A.

Referring to FIGS. 13 to 18, each end 468, 470 of the rigid connector body is shaped to match the shape of the helical path of the bead 143 at the end of each tube segment 402a, 402b. The bead 143 of each tube segment 402a, 402b is a helix. The bead 143 thus comprises a pitch, being the distance between adjacent windings, and a helix angle being the angle of inclination of a winding of the helix from a plane or axis perpendicular to the lumen axis 467A. The bead 143 at the end of each tube segment 402a, 402b, thus comprises inclined end surfaces. The ends 468, 470 of the connector body 462 are shaped to correspond to, and abut, these inclined surfaces. As can best be seen in FIG. 16, the ends 468, 470 comprise helical end surfaces 468A, 470A configured to abut the end of the bead 143. The helix angle of the end surfaces 468A, 470A is substantially the same as the helix angle of the bead 143.

The helix angle of the bead 143 of the first tube segment 402a may be the same as the helix angle of the bead 143 of the second tube segment 402b. Alternatively, the helix angle of the bead 143 of the first tube segment 402a may be different to the helix angle of the bead 143 of the second tube segment 402b.

The helix angle of the helical end surfaces 468A, 470A may be the same.

Where the helix angles of the helical end surfaces 468A, 470A are the same, the helix angles may be the same as the helix angle of one of the two tube segments, or instead may comprise a helix angle which is intermediate the helix angles of the first and second tube segments.

The helix angle of the helical end surfaces 468A, 470A may be different.

Where the first and second tube segment helix angles are different, the helical end surfaces 468A, 470A may each have helix angles corresponding to the helix angle of the adjacent tube segment 402a, 402b.

The pitch of the bead 143 of the two tube segments 402a, 402b may be different. The helix angles of the helical end surfaces 468A, 470A of the intermediate connector may be different.

The rigid connector body 462 of the intermediate connector may be rotationally symmetrical.

The helical end surfaces 468A, 470A, are configured such that they each define an offset portion 468B, 470B that is recessed, towards the centre of the connector body 462 in a direction along the longitudinal axis of the connector body 462, by a distance substantially equal to the diameter of the bead 143. These offset portions 468B, 470B are adjacent bead guides 503, and thus allow the end of the bead 143 to be aligned with the bead guides 503 at the ends of the connector body 462.

Each end 468, 470 of the connector body comprises a plurality of overmould standoffs 501 that project away from the end surfaces 468A, 470A in a direction parallel to the lumen axis 167A.

Adjacent each end 468, 470 is the bead guide 503 which provides an elongate channel 504, to partially receive the end of the bead 143. The film at the end of the bead 143 is cut so that the end of the bead 143 is free to move relative to the remainder of the bead 143. The channel 504 extends partially around the outside of the connector body 462 so as to receive the end of the bead 143, when the bead 143 abuts against, or is at least adjacent, the end surfaces 468A, 470A. The bead guide 503 thus resists movement of the bead 143 in a direction along the lumen axis 167A, and positions the end of the bead 143 so that the wires 151 are adjacent electrical conductors 428 of the intermediate connector 414.

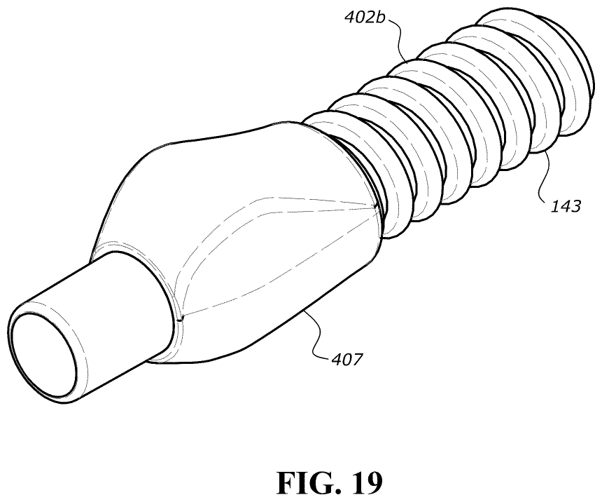

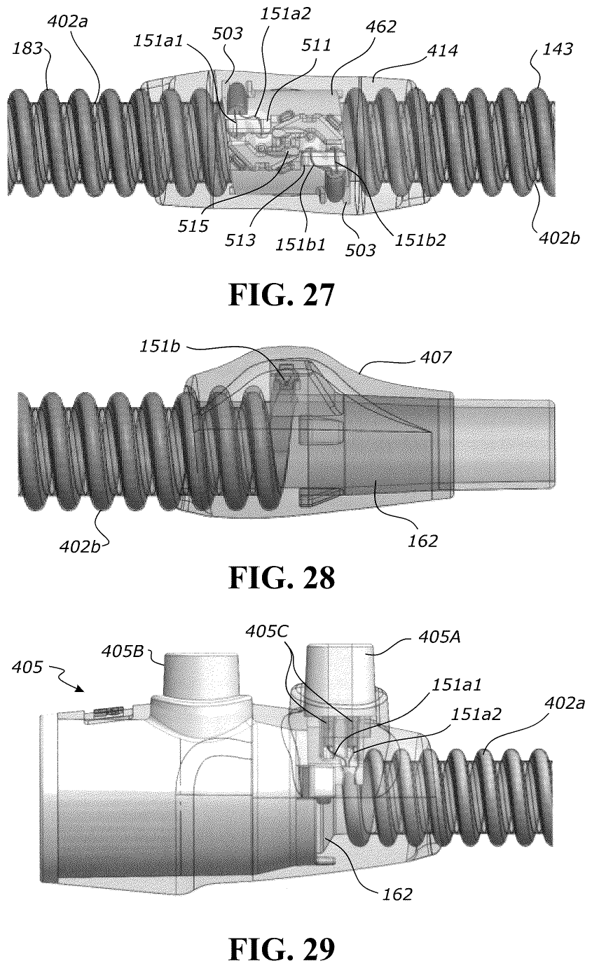

As can best be seen from FIGS. 18 and 27, the electrical conductors 428 form an electrical connection between the wire or wires of first tube segment 402a and the wire or wires of second tube segment 402b.

In some embodiments, the first heater wires 151a can comprise two wires 151a1 and 151a2 and the second heater wires 151b can comprise two wires 151b1 and 151b2. The two wires 151a1 and 151a2 in the first segment 402a can be electrically coupled to the two wires 151b1 and 151b2 in the second segment 402b through electrical conductors 428 on the intermediate connector 414. The electrical coupling creates an electrical path/bridge through the wire 151a1, one of the electrical conductors 428, and the wire 151b1. The electrical coupling creates an electrical path/bridge through the wire 151a2, the other of the electrical conductors 428, and the wire 151b2.

The connector body 462 further comprises two pairs of wire guides 511, 513. One pair of wire guides 511 is associated with connector end 468 and guides the wires 151a1, 151a2 from first tube segment 402a toward the electrical conductors 428. The end of the bead 143 of the first tube segment 402a is received in the bead guide 503 that is adjacent, but spaced from, the wire guide 511. The wire guides 511 comprise tapered portions that guide wires 151a1, 151a2 to corresponding contact areas 428A on electrical conductors 428. The other pair of wire guides 513 is associated with connector end 470 and is electrically connected to the wires 151b1, 151b2 from second tube segment 402b. The end of the bead 143 of the second tube segment 402b is received in the bead guide 503 that is adjacent, but spaced from, the wire guide 513.

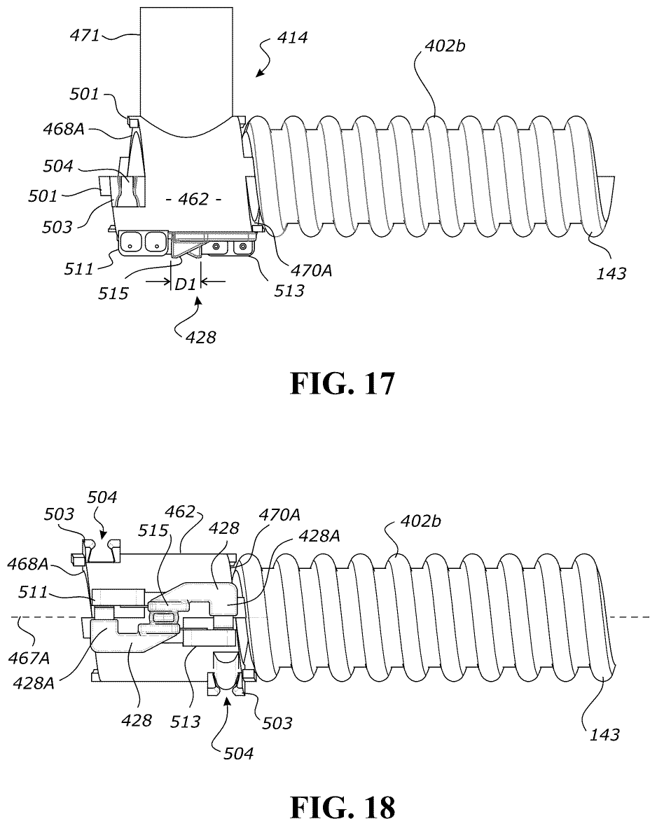

A distance D1, see FIG. 17, between the longitudinally spaced apart wire guides 511, 513 is spanned by an enclosed electrical path along the connector body 462 between the wire guides 511, 513. Distance D1 may be between 3 and 5 mm in one example, optionally about 4.5 mm in another example. Distance D1 may be between 10 and 15 mm in another example, optionally about 13.5 mm.

Each wire guide 511, 513 is circumferentially spaced from its respective bead guide 503 by a distance D2, see FIG. 15. Distance D2 may be between 3 and 5 mm in one example, optionally about 4.2 mm in another example. Distance D2 may be between 15 and 21 mm in another example, optionally about 18 mm.

The wires 151 from each end of the first and second tube segments 402a, 402b extend onto and around the connector body 462 in different, opposed directions, the wires 151a from first tube segment 402a extending clockwise around the connector body 462, the wires 151b from second tube segment 402b anticlockwise, when viewed from one end of the connector body 462, along the lumen axis.

The electrical conductors 428 extend between, wire guides 511, 513. Each wire guide 511, 513 being located at a respective end 468, 470.

The electrical conductors 428 may comprise a plastic component with as many internal electrical paths as are required to connect the number of wires that are present. The electrical conductors 428 may comprise one or more internal wires, and/or one or more other electrical components such as a PCB, or other electrically conductive device.

The electrical conductors 428 may comprise two conductive metallic strips overmoulded together on the rigid connector body 462, to provide a simplified assembly process. Each of the two conductive metallic strips may comprise a solder/contact pad 428A at either end. The intermediate connector body 462 may comprise alignment and connection features within the overmould. The electrical conductors 428 may alternatively be separate components that are permanently or removably mounted on the intermediate connector body 462 during manufacture.

During manufacture, once the wires 151a1, 151a2, 151b1, 151b2 are electrically connected to electrical conductors 428, the wires and electrical conductors 428, and connector body 462 are overmoulded 469. The bead guides 503 help position and retain the bead and wires during manufacture and during use, relative to the connector body 462, and relative to the electrical conductors 428.

The intermediate connector 414 joins first tube segment 402a and second tube segment 402b. The first tube segment 402a can be connected to the device-end connector 405 and the second tube segment 402b can be connected to the patient-end connector 407. When these components are secured together, an electrical path extends from the device-end connector 405 through the first tube segment 402a to the intermediate connector 414, across the intermediate connector 414, and from the intermediate connector 414 through the second tube segment 402b and to the patient-end connector 407. A return electrical path extends through these components in the opposite direction. In effect, an unbroken electrical connection extends from the device-end connector 405 to the patient-end connector 407, and back to the device end connector 405. Similarly, a pneumatic path extends from the device-end connector 405 through the first tube segment 402a to the intermediate connector 414, through the intermediate connector 414, and from the intermediate connector 414 through the second tube segment 402b and to the patient-end connector 407. In effect, an unbroken pneumatic connection extends from the device-end connector 405 to the patient-end connector 407.

The pneumatic path extends from the humidifier chamber 129 connected to the device-end connector 405 through to the patient interface 115 connected to the patient-end connector 407 via wye-piece 409. In effect, these components, namely the first and second tube segments, the intermediate connector 414, the device-end connector 405 and the patient-end connector 407 combine together to define a single medical tube that comprises two or more segments.

The intermediate connector 414 is configured to allow a single controller to control power to the heater wire 151a of first tube segment 402a, wherein the controller can be the humidifier controller 125 as described herein. The humidifier controller 125 controls the heater wire 151a without any additional control functionality located on the intermediate connector 414. This can allow the intermediate connector 414 to be designed using relatively inexpensive components and can reduce the complexity of the design.

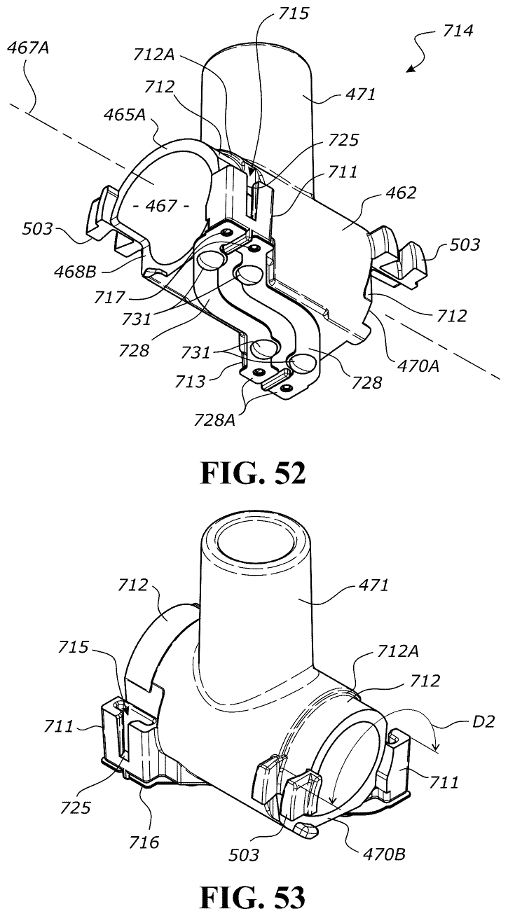

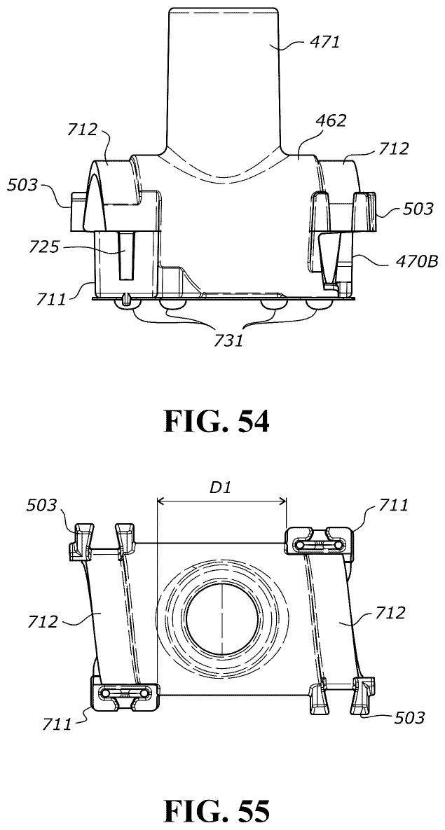

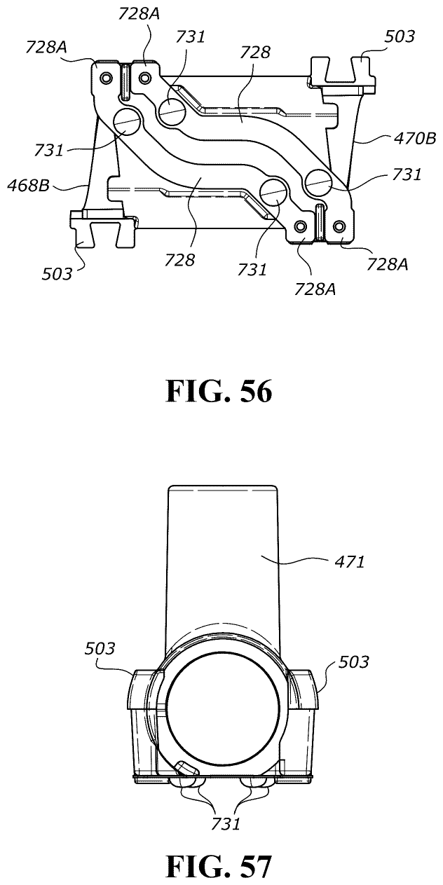

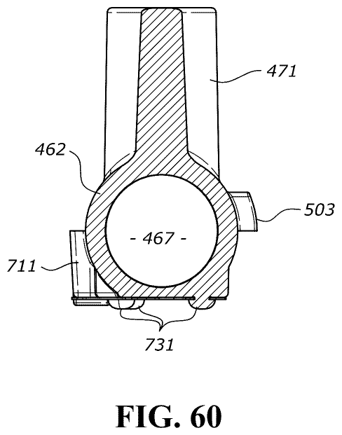

Referring to FIGS. 52 to 68, another intermediate connector 714 is disclosed for a segmented inspiratory limb 402 comprising the medical tube comprising the first segment 402a and the second segment 402b. Intermediate connector 714 has many similar features to connector 414, and like references will be used for like features.

The intermediate connector 714 comprises an internal rigid connector body 462 defining a gas flow lumen 467 comprising a lumen axis 467A. Intermediate connector 714 further comprises a hollow, elongate probe port 471.

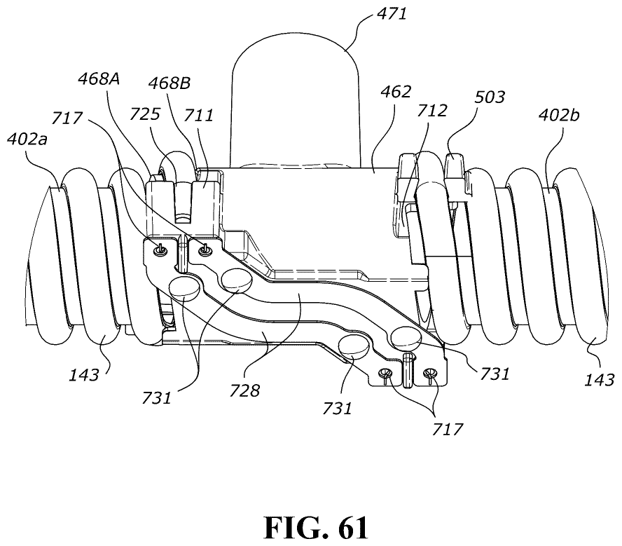

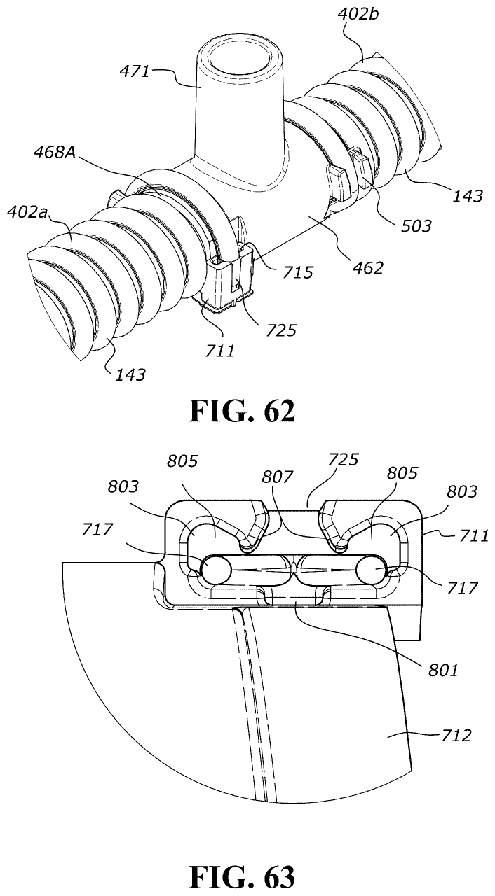

With reference to FIGS. 61 and 62, an end portion of first inspiratory tube segment 402A abuts one end surface 468A of the rigid connector body 462 along offset portion 468B. The end of the first inspiratory tube segment 402A overlaps with, and extends around, the further offset portion 712 (described below). In particular the bead 143 at the end of the first inspiratory tube segment 402A overlaps with, and extends around, the further offset portion 712.

An end portion of second inspiratory tube segment 402B abuts the other end surface 470A of the rigid connector body 462 along offset portion 470B. In particular the bead 143 at the end of the second inspiratory tube segment 402B overlaps with, and extends around, the further offset portion 712 (described below).

The ends of each inspiratory tube segment 402a, 402b, the ends 468, 470 of the connector body 462, and the remainder of the connector body 462 are overmoulded 469 to mechanically and pneumatically connect the tube segments 402a, 402b to the intermediate connector 714.

The ends of the connector body 462, are again configured such that they each define an offset portion 468B, 470B that is recessed, towards the centre of the connector body 462, by a distance substantially equal to the diameter of the bead 143, in a direction along the lumen axis 467A.

Each end of the connector body 462 is defined by a respective helical end surface 468A, 470A that optionally has the same pitch as the offset portions 468B, 470B. The helical end surfaces 468A, 470B, optionally have the same pitch as the bead 143.

The ends of the intermediate connector 714 are also radially inwardly recessed in a direction towards the lumen axis 467A, to provide a further offset portion 712 that functions as a landing for the end of the bead 143, the end of the bead 143 therefore overlapping with the end of the intermediate connector 714. A respective offset portion 712 extends around all or at least part of the periphery of each end of the connector body 462. The further offset portion 712 comprises a channel that is recessed radially inwardly toward the longitudinal axis of the connector body 462, relative to the adjacent portion of the connector body 462. The channel partially receives the bead 143. The channel partially engages the bead 143 to guide the bead 143, and to at least partially restrain the bead 143. For example the channel comprises a base surface on which the underside of the bead 143 rests, and a side wall 712A against which a side portion of the bead 143 abuts. The channel may comprise opposed side walls 712A.

As with intermediate connector 414, the bead guide 503 thus resists movement of the bead 143 in a direction along the lumen axis 167A, and positions the end of the bead 143 so that the wires 151 are adjacent modified electrical conductors 728 of the intermediate connector 714. The bead guide 503 further functions to locate the bead 143 and guide the bead 143 along the offset portion 712 towards the wire guide 711.

As can best be seen from FIGS. 52 and 61, electrical conductors 728 form an electrical bridge being an electrical connection between the first heater wire (which may comprise multiple wires) of first tube segment 402a and the second heater wire (which may comprise multiple wires) of second tube segment 402b.

The connector body 462 further comprises a pair of wire guides 711, 713. One wire guide 711 is associated with connector end 468 and guides the first heater wire comprising wires 151a1, 151a2 from first tube segment 402a toward the electrical conductors 728. The other wire guide 713 is associated with connector end 470 and is electrically connected to the second heater wire comprising wires 151b1, 151b2 from second tube segment 402b. The end of the bead 143 of the second tube segment 402b is received in the bead guide 503 that is adjacent, but spaced from, the wire guide 713.

Each wire guide 711 comprises an oblong, body comprising an inlet opening 715 in the form of an elongate slot configured to receive the wires 151, the wires 151 extending from the end of the bead 143, the end of the bead 143 being restrained and guided by the bead guides 503 and curving, via the offset portion 712, around the arcuate outer surface of the rigid connector body 462 into the opening 715. Opening 715 leads to an internal cavity of the body.

Each wire guide 711, 713 also functions as a bead end retainer, configured to receive, locate and retain the end of the bead 143.

The end 716 of the oblong housing opposite the opening 715 comprises a pair of laterally spaced outlet openings 717 through which respective ends of the wires 151 can project.

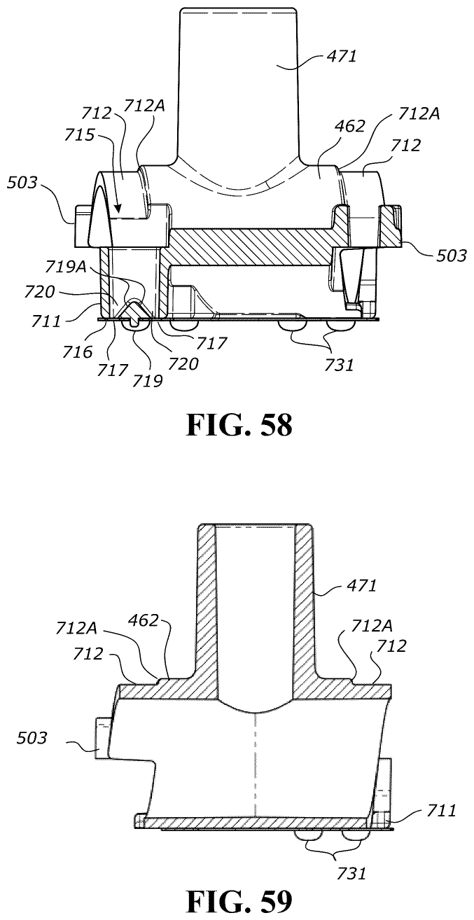

As can be seen in FIGS. 58, 64 and 65, an internal wall comprising a wire deflector 719 is provided at the base 716, between the openings 717.

The wire deflector 719 is triangular and comprises a pair of inclined, planar guide faces 719a that meet at an apex, the apex being towards the inlet opening 715, and aligned with the longitudinal axis of the housing. As the pair of wires 151 are inserted into the wire guide 711 through the inlet opening 715, each wire is guided by a respective guide face 719a towards a respective outlet opening 717. Each outlet opening 717 is laterally spaced from the longitudinal axis. The deflector 719 serves to define discrete and separate tunnels/pathways 720 for each wire 151, inside the wire guide 711. The deflector 719 thus functions to separate the wires 151 during assembly, and also in use. This can be advantageous if the wires 151 attempt to move around due to external forces or heat, the latter occurring when the connector 715 (and tubes) is autoclaved for example.

The wire deflector 719 may have a different configuration, and may for example be semicircular, comprising arcuate guide faces.

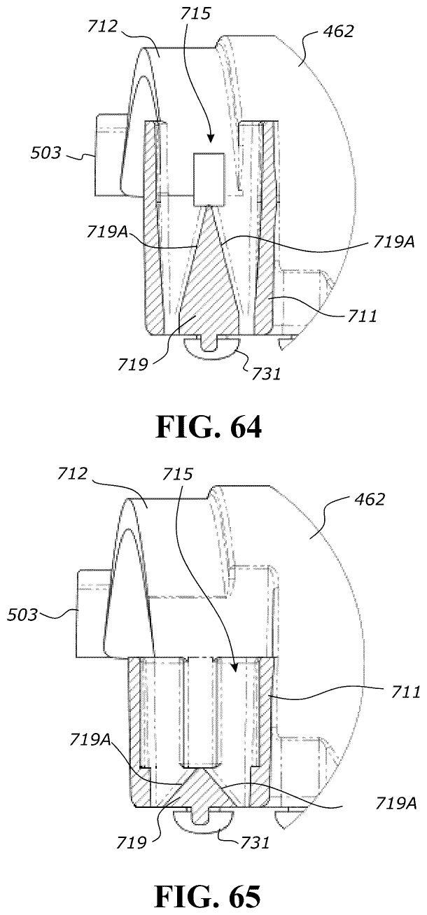

With reference to FIGS. 64 and 65, two different configurations of wire deflector 719 are shown, with one wire deflector 719 being triangular (when viewed from the side) extending relatively far into the cavity of the housing, for example extending over half way into the housing. In this example the wire deflector 79 is about 7 mm long. With reference to FIG. 65, wire deflector 719 is relatively short, and does not project as far into the cavity of the housing, for example extending less than half way into the housing. In this example the wire deflector is about 2 mm long. The wire deflector 719 may project into the cavity of the housing any desired distance that is sufficient to adequately guide the wires 151 into the respective outlet openings 717. For example, the wire deflectors 719 may project between 1 and 10 mm, optionally 2-8 mm, from the outlet openings 717.

With reference to FIGS. 52, 53 and 63, the housing of the wire guide 711 comprises an outmost wall that defines a window 725 to provide a visual guide during manufacture of how far the end of the bead 143, and the wires 151 are pushed into the wire guide 711.

As with intermediate connector 414, intermediate connector 714 comprises a pair of wire guides 711, one at each end of the intermediate connector 714. The wires 151 in one wire guide 711 are electrically connected to the wires 151 in the other wire guide via a pair of electrical conductor elements 728, that extend along the intermediate connector 714, between the wire guides 711.

Each end of each electrical conductor element 728 comprises a tab 728A formed with an aperture 728B through which the end of a respective wire 151 extends, to form an electrical connection with the electrical conductor element 728. This can be seen with reference to FIG. 61. The end of the wires 151 may be secured to the tabs 728A, for example via soldering during the manufacturing process. Note that the solder is not shown in FIG. 61. The electrical conductor elements 728 are secured to the rigid connector body 462 via four protrusions 731 that project from the rigid connector body 462 through respective apertures in the electrical conductor elements 728. The protrusions are heat staked during manufacture to melt the ends of the protrusions to form dome shaped ends that are larger than the apertures in the electrical conductor elements 728, and therefore hold the electrical conductor elements 728 on the rigid connector body 462.

During manufacture, once the wires 151a1, 151a2, 151b1, 151b2 are electrically connected to electrical conductors 728, the wires 151 and electrical conductors 728, and connector body 462 are overmoulded. The bead guides 503, and wire guides 711 help position, restrain and/or retain the bead 143 and wires 151 during manufacture and during use, relative to the connector body 462, and relative to the electrical conductors 728.

The end portions of the first and second heater wires may be overmoulded with the wire guides 711. The overmould material may flow into the housing cavity during manufacture.

With additional reference to FIG. 63, the housing of the wire guide 711 may comprise overmoulding features that facilitate the flow of overmould material into the housing cavity and around the end portions of the heater wires 151.

One overmoulding feature comprises an overmould indentindent 801 at the base of the housing. The overmould indent 801 is, when viewed from above, between the wire openings 717 but adjacent the connector body 462 so as to be spaced away from the window 715. The base of the overmould indent 801, is recessed below the base of the base of the housing of the wire guide, such that overmould material flows into the indent 801 during manufacture, and forms an overmould plug that serves to help retain the overmould material in the cavity of the wire guide 711.

Another overmoulding feature comprises a pair of enlarged corners 803 of the cavity of the wire guide 711, each corner 803 being flared outwardly from the centre of the cavity when viewed from above so as to each define an enlarged space 805 to receive overmould material during manufacture. The enlarged space allows for a greater volume of overmould material to be received in the cavity adjacent each wire opening 717. Each corner 803 is located adjacent a respective side of the window 715, and defines a wing 807 adjacent the window 715 that projects into the cavity. Each wing 807 serves to restrain the overmould material.

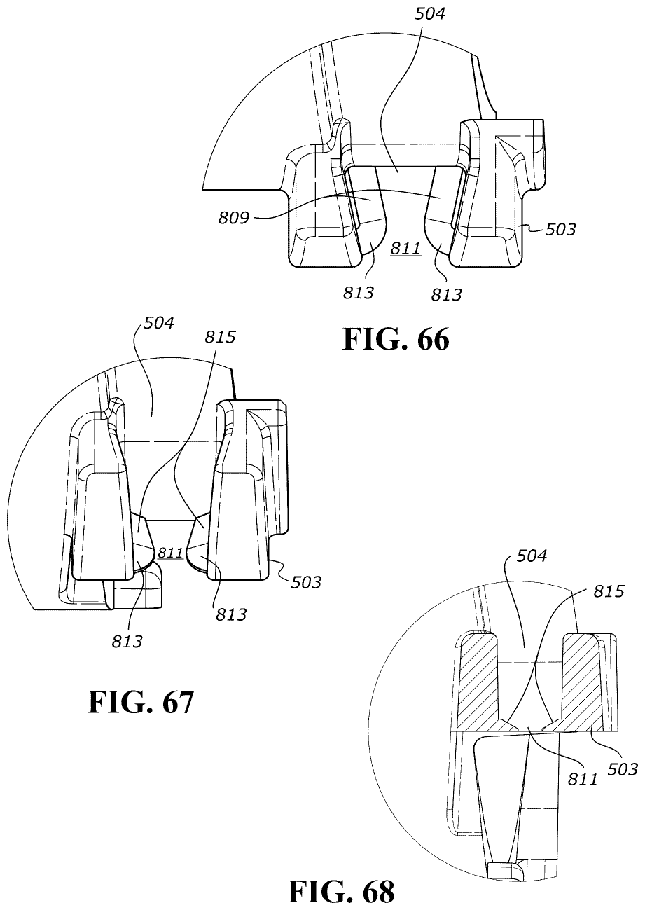

With additional reference to FIGS. 66 to 68, each bead guide 503 may comprise one or more bead engagement features configured to provide an interference fit between the bead 143 and the bead guide 503, to assist in restraining and/or retaining the bead 143 in the elongate channel 504 of the bead guide 503.

The bead engagement features may comprise a pair of opposed protrusions 809 that project from opposed sides of the elongate channel 504, into the elongate channel 504. A gap 811 is defined between the ends of the opposed protrusions 809, the gap 811 being smaller than the resting diameter of the bead 143. The bead 143 is pressed into the gap 811 during manufacture, which deforms the bead 143 such that the protrusions 809 engage with the bead 143 with an interference fit.

Each protrusion 809 comprises a rounded end 813 so as to not to damage the bead 143 during assembly. Each protrusion 809 comprises an inclined upper surface 815 that assists in engaging the bead 143 when the bead 143 is received in the elongate channel 504.

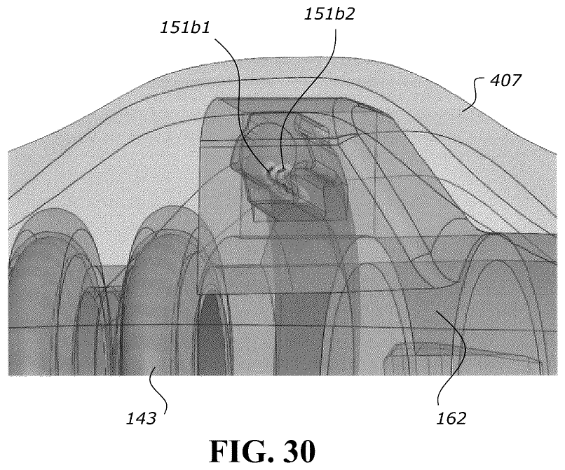

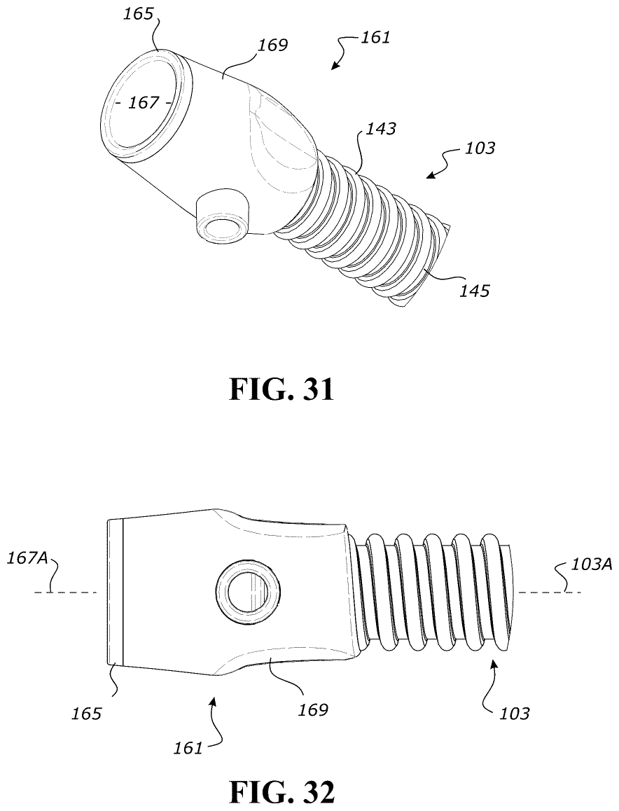

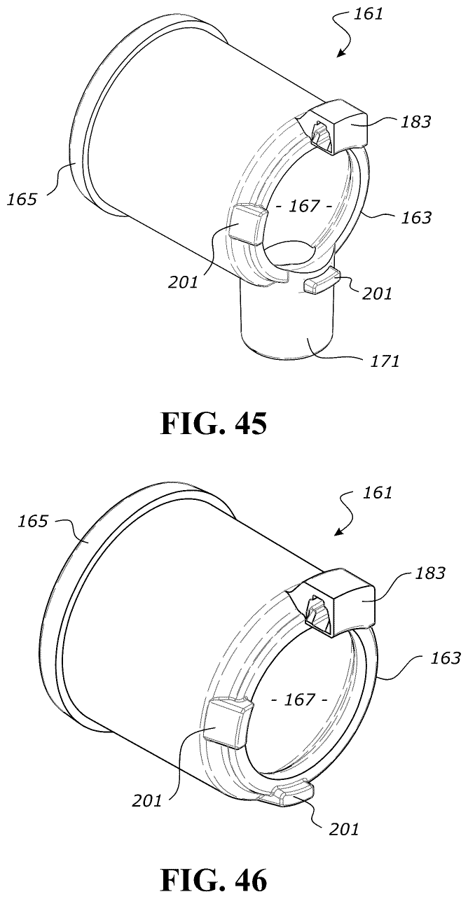

Referring to FIGS. 19, 28 and 30, the patient end connector 407 may be as described with reference to FIGS. 31 to 50, and comprises a connector body 162 comprising a wire retainer 181 and overmould standoffs 201, all overmoulded 169 once the end portion of wire 151 and bead 143 is received in the wire retainer 181. The patient end connector 407 does not comprise a probe port. The patient end connector 407 comprises a male distal end configured to be received in a corresponding female part of the patient interface 115, or of the wye-piece 409. The overmould 169 of the patient end connector 407 is contoured to enable the user to more easily grip, and/or more easily rotate, the patient end connector 407.

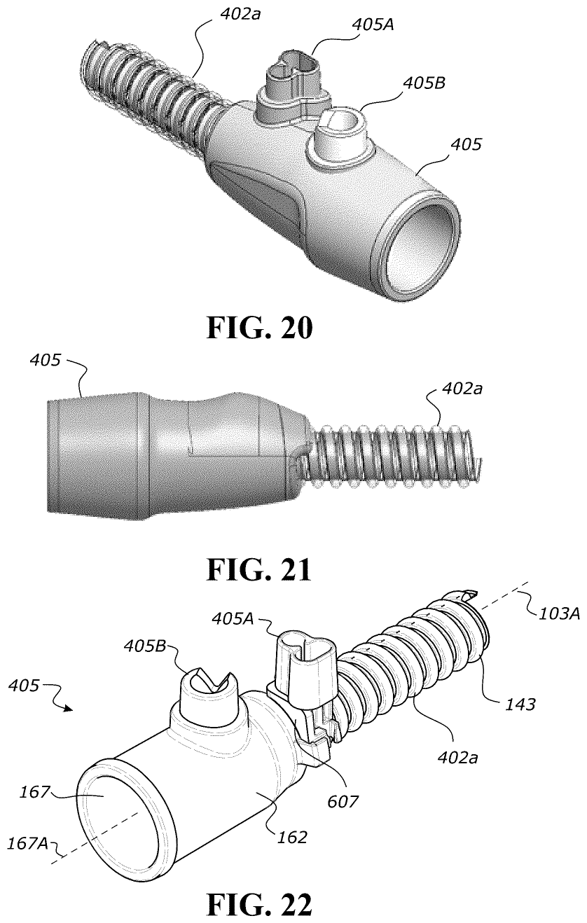

Referring to FIGS. 20 to 26 and 29, the device end connector 405 may be similar to that described with reference to FIGS. 31 to 50, and comprise a connector body 162, overmould standoffs 201, and a probe port 405B formed in a probe port body. No wire retainer 181 is provided. The device end connector 405 comprises a female distal end configured to receive a male part of the device.

The device end connector 405 comprises an electrical connector socket 405A for electrical connection via flying lead to the controller 125. Electrical connector socket 405A may be a separate rigid component, or may be integral with rigid connector 162. Device end probe port 405B receives and locates device end sensor 411.

The electrical connector socket 405A projects in a direction radially outwardly away from the lumen axis 167A.

As can best be seen in FIG. 29, wires 151 project from the end of the bead 143 and extend into respective connector apertures 405C of the socket 405A. Once so connected, the wires and socket 405A are overmoulded. Socket 405A may be only partially overmoulded, that is, only partially along its length, to allow the socket 405A to connect to a corresponding plug.

Consequently, an inspiratory limb 402 is provided comprising:
a) First and second inspiratory tube segments 402a, 402b;
b) A device end connector at one end of first inspiratory tube segment 402a for connecting first tube segment to a device that supplies heated, humidified breathable gases to the first tube segment 402a;
c) An intermediate connector connecting the other end of first tube segment 402a to a first end of second tube segment 402b;
d) A patient end connector at the other end of second tube segment 402b, for connecting second tube segment 402b to a patient interface.

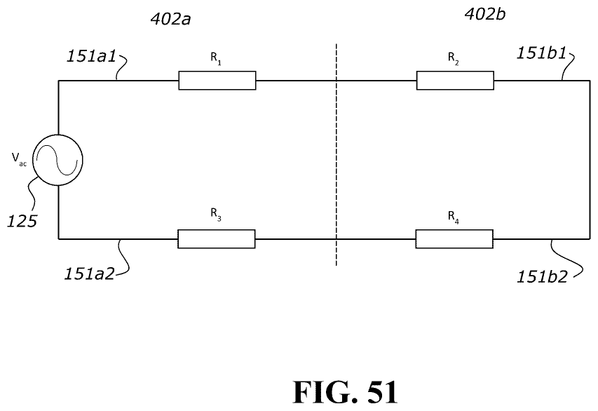

The system 401 may comprise:
a) an inspiratory limb 402 which comprises first and second inspiratory tube segments 402a, 402b, each of which comprises one or more heater wires, wherein the one or more heater wires are electrically connected at the intermediate connector 414;
b) a controller which provides electrical power to the first and second tube segments in series, the first tube segment 402a being the only tube segment directly connected to, and receiving electrical power directly from, the controller. Such a circuit can be seen with reference to FIG. 51.

The electrical power used by the second tube segment 402b is determined by the electrical properties of the first and second tube segments 402a, 402b.

The inspiratory circuit comprises:
a) A device end sensor connected to the controller to allow the controller to determine the temperature of breathable gases at the device end connector 405.
b) An intermediate sensor at the intermediate connector 414 and connected to the controller to allow the controller to determine the temperature of breathable gases at the intermediate connector 414, that is, at the downstream end of the first tube segment 402a, and the upstream end of the second tube segment 402b.
c) No sensor at the patient end connector 407.
d) No sensor on the second tube segment 402b.

The system 401 may be configured to control the heating provided by the inspiratory limb 402 using closed loop control for the tube segment(s) outside of the temperature controlled environment, and using open loop control for the tube segment(s) inside the temperature controlled environment.

The inspiratory limb of the current disclosure may be used where the patient is in a temperature controlled environment, such as an incubator 308. The temperature inside an incubator is typically between 31-37° C., although may sometimes be lower in practice. The operating (target) temperature range for gases in the inspiratory limb may be between 31-40° C.

The inspiratory limb may be configured such that:
a) the first inspiratory tube segment 402a is outside the temperature controlled environment, and may therefore be in the ambient environment. The ambient environment may comprise a room or space in a building in which the incubator 308 is located.
b) The second inspiratory tube segment 402b is inside the temperature controlled environment, in this example, the incubator 308.
c) All of the sensors 411, 413 of the inspiratory limb are located outside the temperature controlled environment. The intermediate sensor 413, at the downstream end of the first tube segment 402a, and the intermediate connector 414, is located adjacent, but outside, the temperature controlled environment. The sensors 411, 413 are therefore measuring temperature of the breathing gases in parts of the inspiratory limb that are outside the temperature controlled environment.

In an embodiment, the controller 125 may be configured to control the system to:
a) Deliver breathable gases to the upstream end of the first tube segment 402a at first temperature, for example a temperature of around 37° C.
b) Increase the temperature of the breathable gases by a first predetermined amount to a second temperature, for example to around 40° C., at the intermediate connector 414, that is, just prior to entering the incubator 308.

Such heating control, of the part of the inspiratory limb outside of the incubator 308, may be via closed loop control.

One or more of the tube properties of the inspiratory limb are such that the system 401 may be configured to:
a) increase the temperature of the breathable gases by a second predetermined amount to a third temperature, for example to less than 42° C., in the second tube segment 402b; and/or
b) maintain the temperature of the breathable gases, from the intermediate connector 414 to the patient end connector 407; and/or
c) prevent a decrease in temperature below 33° C.

Such heating control, of the part of the inspiratory limb in the temperature controlled environment (for example inside the incubator 308), may be via open loop control.

Such heating control may be achieved under predetermined ambient conditions and/or for predetermined flow rates. For example, such control may be provided when the ambient temperature is between 2° and 26° C. and/or when the gases flow rate is between 0.5 and 40 L/min.

The controller 125 cannot independently provide electrical power to each of the primary tube segment 402a and the secondary tube segment 402b. The heat power output of each segment 402a, 402b of the inspiratory limb is determined by the physical and/or electrical characteristics of each segment 402a, 402b, as well as the electrical power supplied directly to primary tube segment 402a from the electrical power source, namely the controller 125. The heater wires in the first and second tube segments 402a, 402b are connected in series, always powered together, and use the same electrical current.

The controller 125 is electrically connected to the first tube segment 402a to provide electrical power to the inspiratory limb 402. A proportion of that delivered electrical power is converted to a first heat power output from the first tube segment 402a. The first tube segment 402a is electrically connected to the second tube segment 402b. A proportion of that delivered electrical power is converted to a second heat power output by the second tube segment 402b.

The amount of electrical power that is used by the second tube segment 402b is determined by the relative electrical properties of the first and second tube segments 402a, 402b. Examples of these electrical properties include the resistance of each tube segment 402a, 402b.

The resistance of each tube segment 402a, 402b may be determined by one or more physical properties of each tube segment 402a, 402b. These properties may include any one or more of:
a) wire gauge, that is, wire diameter;
b) wire material;
c) wire length;
d) if the wire is helically wound, any one or more of:
   i. the helix pitch;
   ii. the helix angle;
   iii. the helix length;
   iv. the helix diameter;
   v. the number of helix windings.

The heat power output from each tube segment 402a, 402b may be determined by the amount of electrical power received by each tube segment 402, 402b, and also by physical properties of each tube segment 402a, 402b. These physical properties can include any one or more of:
a) tube segment material;
b) tube segment length;
c) tube segment inner diameter (i.e. lumen diameter);
d) tube segment insulation;
e) tube segment bead material;
f) tube segment bead diameter;
g) tube segment bead length;
h) tube segment bead helix pitch;
i) tube segment bead helix angle;
j) tube segment bead helix length;
k) the number of tube segment helix windings.

The physical and/or electrical properties of the first and second tube segments 402a, 402b may be configured such that between 3-20%, and optionally 3-12%, of the total power delivered to the inspiratory circuit by the controller 125 is used by the downstream second tube segment 402b. Any higher than 20% and the second tube segment 402b could significantly raise the gas temperature and lead to an unsafe tube, because the breathable gas is too hot for the patient to safely breathe, and/or because the exterior surface of the second tube segment 402b is too hot for the patient and/or user to safely touch. Any lower than 3% and the second tube segment 402b could be subject to unwanted formation of condensate in the tube segment 402b.

The medical tube of the inspiratory limb of the breathing circuit may comprise any one or more of the following characteristics:
a) the length of the first inspiratory segment 402a may be between 1100-1400 mm, optionally 1300 mm;
b) the length of the second inspiratory segment 402b may be between 200-400 mm, optionally 300 mm;
c) the diameter of the wire in the first inspiratory segment 402a may be between 0.1 and 0.3 mm, optionally approximately 0.2 mm;
d) the diameter of the wire in the second inspiratory segment 402b may be between 0.2 and 0.5 mm, and optionally approximately 0.3 mm;
e) the resistance of the heater wire of the inspiratory circuit may be between 10 and 25Ω, and optionally is approximately 17;
f) the resistance of the heater wire of the first inspiratory segment 402a may be between 10 and 25Ω, and optionally is approximately 15Ω;
g) the resistance of the heater wire of the second inspiratory segment 402b may between 0.5 and 3Ω, and optionally is approximately 1Ω.

The first segment 402a, for example, can have a length that is at least about 0.5 m and/or less than or equal to about 2 m, at least about 0.7 m and/or less than or equal to about 1.8 m, at least about 0.9 m and/or less than or equal to about 1.5 m, or at least about 1 m and/or less than or equal to about 1.2 m, or at least about 1.2 m and less than or equal to 1.3 m.

The second segment 402b, for example, can have a length that is at least about 0.2 m and/or less than or equal to about 1.5 m, at least about 0.3 m and/or less than or equal to about 1 m, at least about 0.4 m and/or less than or equal to about 0.8 m, or at least about 0.5 m and/or less than or equal to about 0.7 m; or at least about 0.25 m and/or less than 0.35 m, and optionally at least about 0.28 m and/or less than 0.32 m.

Alternative embodiments may comprise wire with the same diameter in each tube segment 402a, 402b but different resistance values, and/or different wire helix pitch in each tube segment 402a, 402b, and/or a different number of wires in each tube segment 402a, 402b. The bead pitch can also be varied, and this may also have an effect on the flexibility of the tube segment 402a, 402b.

One or both tube segments 402a, 402b may comprise a variable wire helix pitch along some or all of the length of the segment 402a, 402b. For example, the wire helix pitch at the patient end of the second tube segment 402b can be configured to increase the flexibility of the tube segment 402b over a portion of its length, as compared to the flexibility along the remainder of its length. The flexibility of one tube segment 402a, 402b, may be different to that of the other tube segment 402a, 402b. The different flexibilities may be achieved via each tube segment 402a, 402b having a different wire helix pitch along its length. One tube segment 402a 402b may comprise a wire wound at a different winding density to that of the other tube segment 402a, 402b.

TABLE A

| | | | | | | | | | % |
|---|---|---|---|---|---|---|---|---|---|
| | | | Inspiratory | Extension | | | | | Extension |
| Inner | Inspiratory | Extension | Wire | Wire | | Insulation | Inspiratory | Extension | Power of |
| Diameter | Length | Length | Diameter | Diameter | Pitch | Thickness | Resistance | Resistance | Total |
| (mm) | (mm) | (mm) | (mm) | (mm) | (mm) | (mm) | (Ω) | (Ω) | Power |
| 15 | 1100 | 350 | 0.26 | 0.36 | 3.2 | 0.2 | 11.49 | 1.99 | 14.78 |
| 12 | 1000 | 300 | 0.15 | 0.33 | 5.3 | 0.2 | 15.48 | 1.00 | 6.10 |

Table A above discloses some example tube properties, in accordance with this disclosure. Reference to ‘inspiratory’ are to the first tube segment 402a, and to ‘extension’ are to second tube segment 402b.

In some embodiments, the power source provides rectified AC or DC power to the heater wire 151a. In some embodiments, the amount of power provided can be adjusted by adjusting a duty cycle of power applied to the heater wire 151a in the first tube segment 402a. For example, pulse-width modulation (PWM) can be used to power the heater wire 151a and the duty cycle of the PWM signal can be adjusted to control the power delivered. In another example, the amount of power used by the heater wire 151a can be adjusted by controlling the amplitude of the power signal.

The controller 125 can use the readings from the inspiratory limb sensors 411, 413 to adjust power to the heater wires 151, using, for example pulse-width modulation.

The sensor 413 is positioned at the connection or intersection between the first and second segments 402a and 402b to provide to the controller 125 with a signal indicative of a parameter of gases entering the second tube segment 402b, which is indicative of the parameter of gases entering an incubator or other such temperature controlled region having a different temperature from the ambient environment. The controller 125 can use these readings to adjust the electrical power output to the inspiratory limb 402.

The target or suitable temperature can vary depending at least in part on the application and environment it is being used in, and can be about 37° C., about 40° C., at least about 37° C. and/or less than or equal to about 38° C., at least about 36.5° C. and/or less than or equal to about 38.5° C., at least about 36° C. and/or less than or equal to about 39° C., at least about 35° C. and/or less than or equal to about 40° C., at least about 37° C. and/or less than or equal to about 41° C., or at least about 39.5° C. and/or less than or equal to about 40.5° C. The delivered temperature can vary depending at least in part on the flow rate of gases. For low flow rates, for example 0.51/m, the end of hose temperature may be higher. For low flow rates, more electrical power may be provided to the humidifier chamber and to the heater wire, as the gases spend more time in the system and therefore have more time to cool down. The delivered temperature is the temperature of gases delivered to the patient interface, and can be referred to as the ‘end of hose’ temperature. The delivered temperature can also vary depending on the ambient environment.

The controller 125 can control operation of various other components of the respiratory humidification system 301.

While the illustrated system is illustrated as using a single controller 125, multiple controllers can be used in other configurations. The multiple controllers can communicate or can provide separate functions and, therefore, the controllers need not communicate. In some implementations, the controller 125 may comprise a microprocessor, a processor, or logic circuitry with associated memory or storage that contains software code for a computer program. In such implementations, the controller 125 can control operation of the respiratory humidification system 401 in accordance with instructions, such as contained within the computer program, and also in response to internal or external inputs. The controller 125, or at least one of the multiple controllers, can be located with the breathing circuit, either attached to the breathing circuit or integrated as part of the breathing circuit.

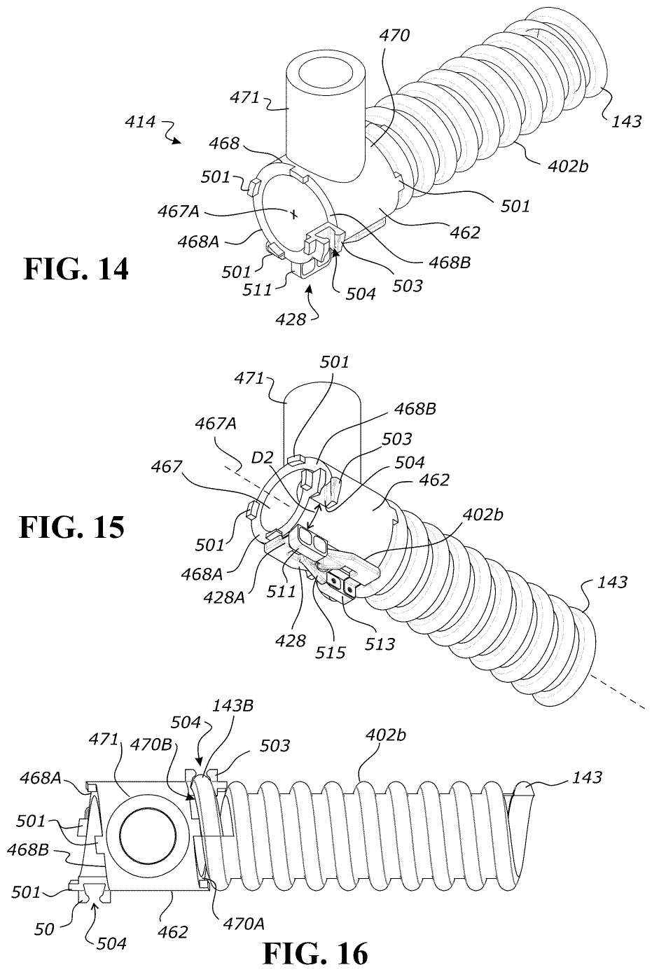

One or both end connectors 405, 407 may, for example, be an end connector similar or identical to, or having some or all features of, the end connectors described below with reference to, FIGS. 14 to 24.

The medical tube comprises the tube segments 402a, 402b. These tube segments 402a, 402b may be releasably connected together via the intermediate connector 414. Alternatively, the tube segments 402a, 402b may be permanently connected together.

As can best be seen with reference to FIG. 33, end connector body 162 is shown having a first end 163 attached to one end of, for example, an inspiratory tube 103, the tube 103 comprising a helically wound bead 143 in which at least one wire 151 is embedded. The end of the tube 103 is connected to the first end 163 of the connector body 162 via the overmould 169 which covers the connector body 162 and the end of the tube 103.

As described above, one or more medical tubes in a typical breathing circuit may comprise one or more sensors in an end connector 161 of the medical tube. For example, an inspiratory tube 103 may comprise a probe/sensor port 171 in one or both end connectors 161. One or more other medical tubes in a typical breathing circuit may not comprise any sensors.

Where the medical tube is provided with one or more wires 151 as described above, for example heating and/or sensor wires, it is desirable that the, or each, wire 151 remains in the desired position and orientation in the medical tube. The or each wire 151 may remain substantially in place along the length of the medical tube, where the or each wire 151 is embedded in a bead 143 of the tube as described above with reference to the FIG. 2. The end portion 151C of the, or each, wire 151 is the portion that extends from the end of the bead 143. The end portion 151C of the wire 151 could simply be the terminal end of the wire 151, or could comprise a loop of wire where the wire is looped back and extends back into the bead 143, or could comprise two wires 151 that are twisted, soldered or otherwise connected together and optionally folded underneath the bead 143.

It can be possible that one or more of the wires 151 can move during manufacture, and/or during use. For example, an end portion of a wire 151 encapsulated within a reinforcing bead 143 of a medical tube may move and/or extend when the medical tube undergoes assembly or reprocessing. Reprocessing is where the medical tube undergoes a further process, such as autoclaving, after initial use, for example to clean and/or sterilise the medical tube. Such processes typically use heating/cooling and/or pressure changes.

Provided herein is a tube end connector 161 for a respiratory tube 103 which comprises a wire retainer 181 configured to reduce movement and/or extension of an end portion of at least one wire 151 that extends from one end of the tube 103.

With reference to FIGS. 33-36, the wire retainer 181 comprises a housing 183 comprising a mouth 185 leading to an internal cavity 187 configured to receive an end portion of wire 151 such that some or all of the end portion of the wire 151 is contained in the cavity 187. The housing 183 therefore comprises an enclosure for the end portion of the wire 151 to restrain the end portion of the wire 151. The housing 183 is configured to reduce movement of the wire 151, and/or resist extension of the wire 151 from the end of the bead 143. The housing 183 may also retain and restrain an end portion of the bead 143.

In this embodiment, the housing 183 entirely encloses the cavity 187, with the only opening into the cavity 187 being mouth 185. The cavity 187 therefore comprises a base 191, opposed side walls 193 and end wall 195 upstanding from the base 191, and a top 197. The cavity 187 is elongate and comprises a longitudinally extending cavity axis 187A which extends from end wall 195 to opposed mouth 185. The cavity 187 may be substantially cuboidal.

The housing 183 comprises a housing block that projects outwardly from the connector body 162.

The end wall 195 is configured to provide an end stop for the wire 151 preventing the wire 151 from extending into the housing 183 beyond the end wall 195. The end wall 195 may be configured such that the end portion of the wire contacts the end wall 195, folds back on itself, and extends back towards or through mouth 185.

In this embodiment the housing 183 is oriented such that the housing axis 183A is substantially orthogonal to the lumen axis 161A of end connector 161. The housing 183 therefore extends laterally across, and projects from, the end of the connector body 162. In this embodiment the housing 183 is provided adjacent first end 163 of the connector body 162.

With additional reference to FIGS. 12 to 18, the bead 143 is helically wound about the tube 103. The bead 143 may be helically wound around the outside of the film 145. The film 145 may itself be helically wound. The film 145 may be helically wound around the outside of the bead 143. The film 145 may solely form the lumen, or the bead 143 and film 145, in combination may form the lumen.

At the end of the bead 143, at first end 163 of the connector body 162, the end portion 143B of the bead 143 projects from the end of the tube 103, but continues, to some extent, to adopt a helical form such that the bead end portion 143B projects in arcuate form from the tube 103, when viewed along the lumen axis 167A. In the orientation of the connector 161 shown in FIGS. 33 to 36, the bead end portion 143B projects outwardly away from the lumen of the tube 103 and then arcs towards the housing 183 such that the bead end portion 143B is spaced from the tube 103, and extends in a direction substantially orthogonal to the lumen axis 167A into the housing 183.

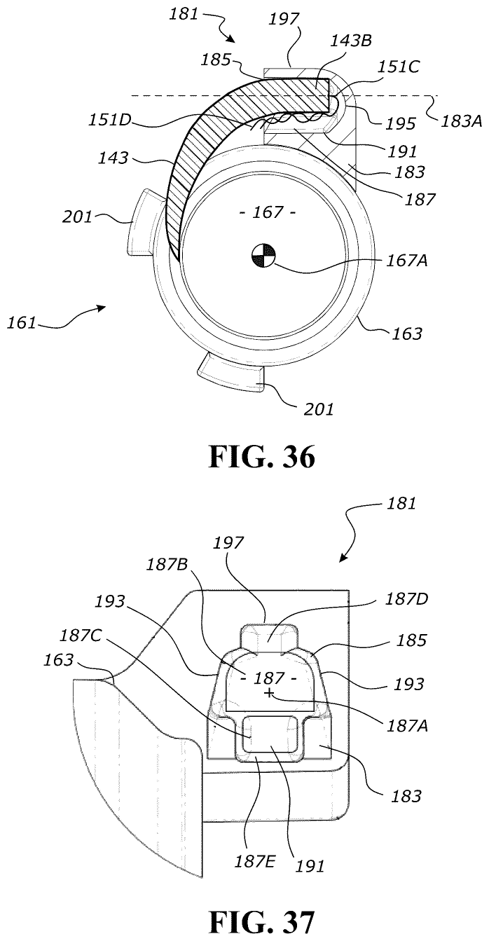

The housing 183 is also spaced from the exterior of the tube 103, with the housing axis 183A substantially aligned with the axis of the bead end portion 143B, and with the mouth 185 facing the bead end portion 143B. The bead end portion 143B extends through the mouth 185 into the housing cavity 187. The end portion 151C of the wire 151 is therefore contained within the cavity 187, with the end portion 151C abutting the end wall 195. End wall 195 is configured such that the end portion 151C may abut or be adjacent to the end wall 195 and then extend back towards, and optionally through, the mouth 185. The terminal end 151D of wire 151 may project from the housing 185, as can be seen from FIG. 36. The housing 183, mouth 185, and cavity 187 are therefore oriented relative to the connector lumen axis 167A, and relative to the bead 143, to be able to receive the bead end portion 143B, and the end portion 151C of the wire 151 without bending or further deformation of the bead end portion 143B and end portion 151C. The axis 187A of cavity 187 is therefore aligned with the axis of the bead end portion 143B.

Referring to FIGS. 36 and 37, the cavity 187 of the housing 183 may be configured to provide a larger cavity portion 187B configured to receive the bead end portion 143B and any overmould material, and a smaller adjacent cavity portion 187C in which the end portion 151C of the wire 151 can be folded or bent back under the bead end portion 143B after having abutted cavity end wall 195. The cross-sectional area of larger cavity portion 187B may be greater than the cross-sectional area of smaller cavity portion 187C, when viewed along the cavity axis 187A. The upper part of larger cavity portion 187B may be arcuate, so as not to comprise straight edges. The smaller cavity portion 187C may be substantially oblong in cross section.

When viewed along the cavity axis 187A, in one or more examples:
a) The larger cavity portion 187B, in one example, may be approximately 3.6 mm wide by 2.0 mm high, or approximately 4.0 mm wide by 2.0 mm high.
b) The smaller cavity portion 187C, may be approximately 1.7 mm wide by 1.3 mm high, or approximately 2.0 mm wide by 1.0 mm high.
c) The larger cavity portion 187B may be between 1.5 and 2.5 times wider than smaller cavity portion 187C, optionally between 1.75 and 2.5 times wider, optionally between 1.2 and 2.5 times wider, optionally approximately 2 times wider.
d) The larger cavity portion 187B may be between 1.1 and 2.0 times taller than smaller cavity portion 187C, optionally between 1.25 and 1.85 times wider, optionally between 1.2 and 1.9 times, optionally approximately 1.5 times wider.

Cavity 187 may comprise an uppermost cavity portion 187D, of smaller cross section that larger cavity portion 187B, and which provides an overflow space to accommodate variation in the size of the bead end portion 143B and/or any overmould material that may be present.

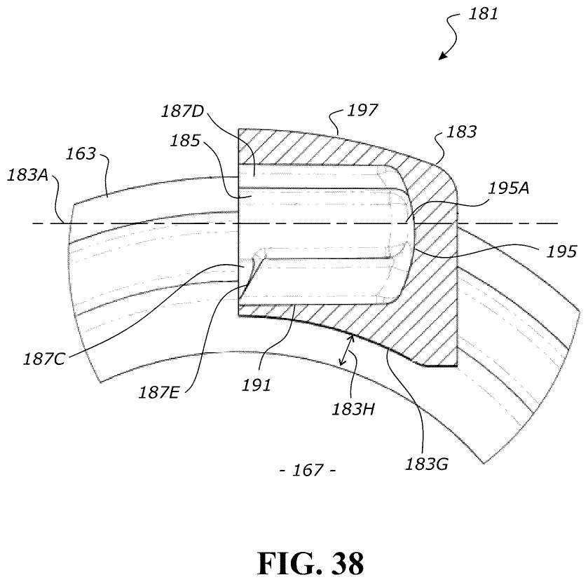

The mouth 185 may comprise one or more lead-in formations, configured to guide the bead end portion 143B and/or wire end portion 151C into the cavity 187. With reference to FIG. 38, the lower cavity portion 187C comprises a lead-in formation comprising an inclined ramp 187E, that extends from a lower margin of the mouth 185, upwardly into the cavity 187. The ramp 187E may be inclined at an angle of between 5 and 85 degrees, optionally 10-45 degrees, in one example about 60 degrees to planar cavity base 191.

The housing cavity 187 may be around 5 mm long, from mouth 185 to end wall 195. The longitudinal distance between the inner end of ramp 183E and the inner end of arcuate end wall 195 may be around 3 mm.

The end wall 195 of the cavity 187 is configured to limit further movement of the bead end portion 143B and/or the wire end portion 151C by abutting the bead end portion 143B and/or the wire end portion 151C if necessary. The end wall 195 prevents movement of the bead end portion 143B and/or the wire end portion 151C relative to the connector body 162 during use.

With reference to FIG. 38, the end wall 195 is arcuate when viewed perpendicular to cavity axis 187A, the end wall 195 being concave in this example. Arcuate end wall 195 allows wire end portion 151C to bend back on itself, such that the end portion of the wire 151C does not project into, or get stuck in, end wall 195.

With reference to FIG. 38, the arcuate end wall 195 also provides a clearance 195A between itself and the bead end 143B, that clearance 195A providing a space to accommodate some or all of the wire end portion 151C.

With reference to FIG. 38, the housing 183 may comprise an external base surface 183G that is radially outwardly spaced 183H from the connector lumen 167. Space 183H provides a clearance for overmould material that may flow underneath the housing 183 between the housing 183 and the lumen 167.

As can be seen with reference to FIGS. 34 and 35, the housing 183 is provided at the first end 163 of the end connector body 162. The housing 183 projects radially outwardly from the end connector body 162 and lumen axis 167A, and also projects longitudinally outwardly away from the first end 163 in a direction substantially parallel with the lumen axis 167A.

With further reference to FIGS. 34 and 35, the end connector body 162 in this embodiment further comprises a plurality of overmould standoffs 201 in the form of lugs that project radially outwardly from the end connector body 162, and which also project longitudinally outwardly from the first end of the end connector body 162. The underside of each overmould standoff 201 is radially outwardly spaced from the lumen, such that there is a clearance between an underside of the standoff 201 and the lumen, when viewed along the lumen axis, see FIG. 36. The end of the tube 103 is also spaced from the end of the connector body 162. This space provides a clearance to allow overmould material to flow underneath the overmould standoffs 201 and into the space between the end of the tube 103 and the end of the connector body 162, such that the overmould material forms part of the lumen between the tube 103 and connector body 162.

The overmould standoffs 201 also function as overmould anchors on the connector 161 which are encapsulated by the overmould material during manufacture and which assist in securing and retaining the overmould material on the connector 161.

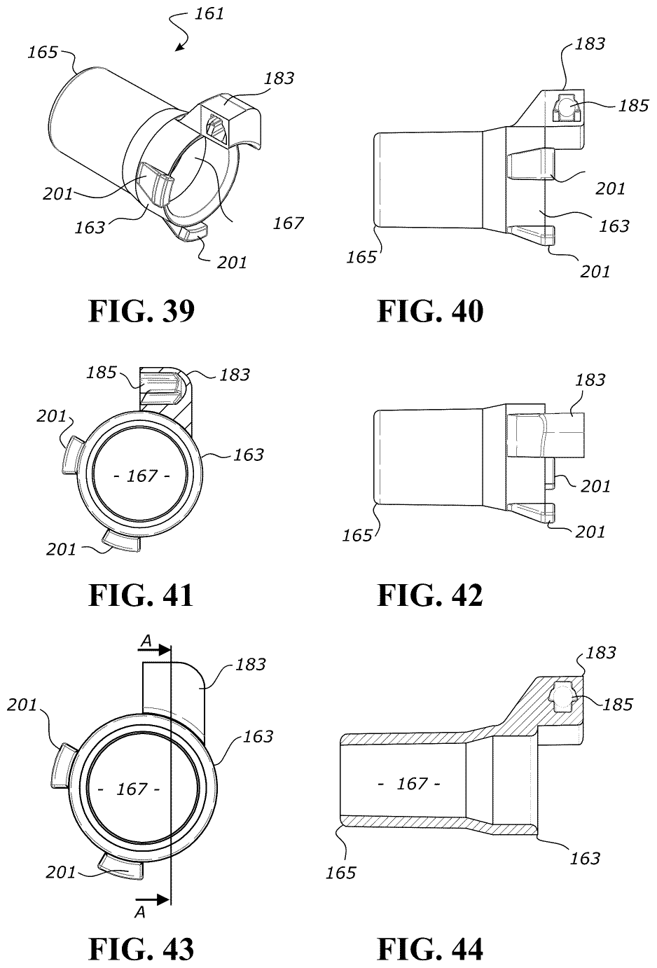

With reference to FIGS. 39 to 50, various tube end bodies 162 are shown and comprise the above described features, including the wire retainer.

Each connector body 162 can, when overmoulded as described above, form a tube connector 161.

Each connector 161 can be configured as a male connector or as a female connector, as required. Each connector 161 is described for use in a particular application (e.g. neonatal, adult). However, for each connector 161, other, or different applications, are envisaged.

It is envisaged that the described connectors 161 can be used with tubes that differ from those described. This disclosure is not limited to the specific tube types and constructions described herein.

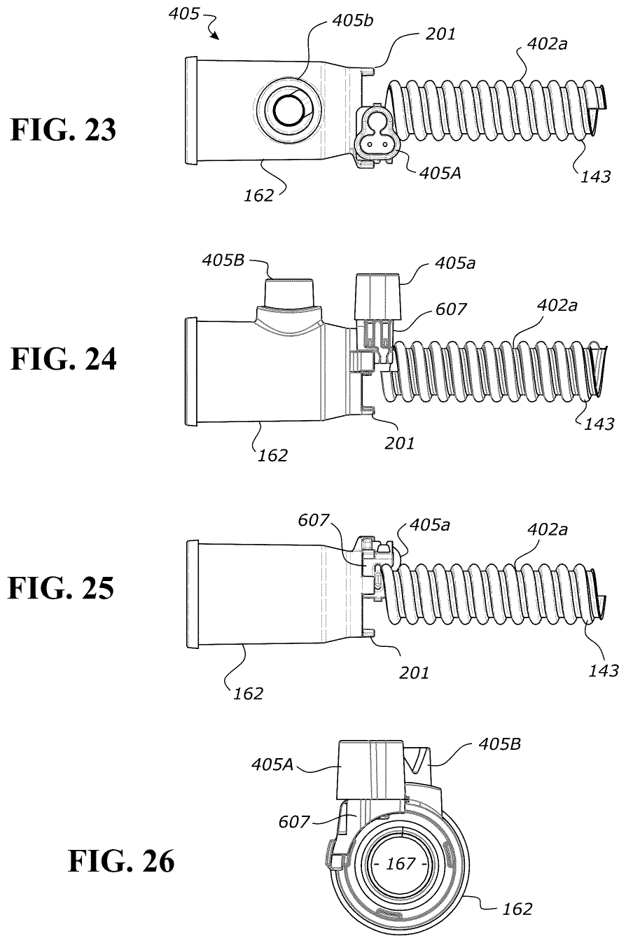

With reference to FIG. 21, end connector body 162 is configured for use with a neonatal tube. In this example, second end of connector body 162 comprises a male connector. This connector body 162 comprises two overmould standoffs 201.

With reference to FIGS. 39 to 44, end connector body 162 is configured for use with an adult tube. In this example, second end of connector body 162 comprises a female connector. This connector body 162 comprises two overmould standoffs 201. This connector body 162 comprises a probe/sensor port 171.

With reference to FIG. 46 end connector body 162 is configured for use with an adult tube. In this example, second end of connector body 162 comprises a female connector. This connector body 162 comprises two overmould standoffs 201. This connector body 162 does not comprise a probe/sensor port.

In each of the examples of FIGS. 39 to 46, the housing 183 of the wire retainer 181 comprises a cavity 187 enclosed on all sides, with access to the cavity 187 for the wire end portion 151A only being via mouth 185. In these examples, the housing 183 resists movement of the wire end portion 151C in all directions away from the cavity axis 187A, and in one direction along the cavity axis 187A (namely in the direction towards end wall 195). The housing 185 thus constrains movement of the wire end portion 151C all directions apart from one direction, that is along cavity axis 183B towards mouth 185.

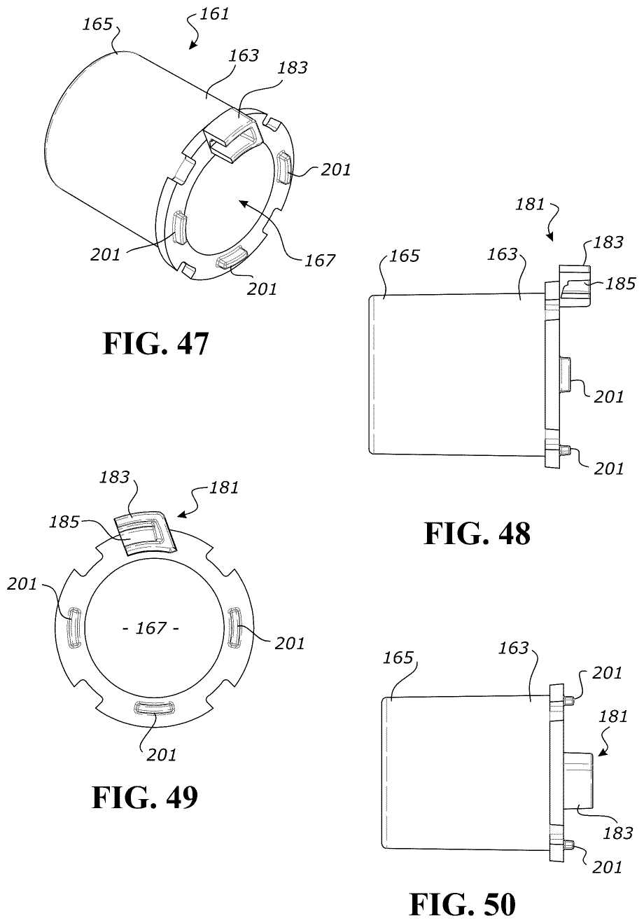

With reference to FIGS. 47 to 50, a connector body 162 is shown for use with a dryline adult tube. In this embodiment a wire retainer 181 is provided comprising a housing 185 in which the top wall is missing. In this embodiment, the housing 183 of the wire retainer 181 comprises a cavity 187 enclosed in all directions apart from two, with access to the cavity 187 for the wire end portion 151A only being via mouth 185 and the top of the housing 185.

In these examples, the housing 183 resists movement of the wire end portion 151C in all directions away from the housing axis 183A apart from away from the bottom of the housing along the lumen axis 167A, and in one direction along the cavity axis 187A (namely in the direction away from end wall 195). The wire end portion 151C thus has freedom of movement in only two directions, that is along cavity axis 187A towards mouth 185, and radially outwardly away from the housing base.

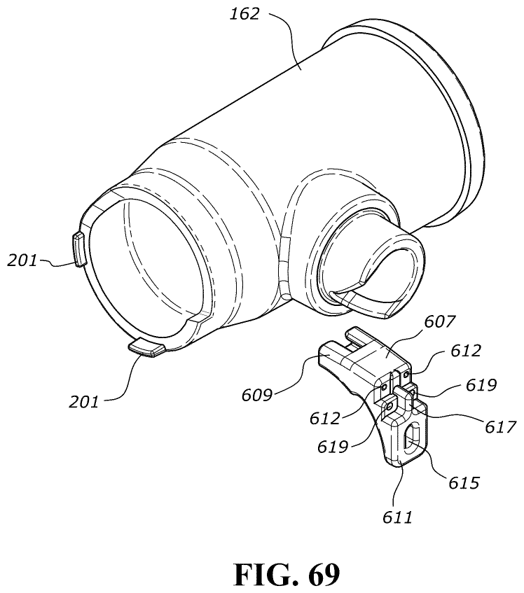

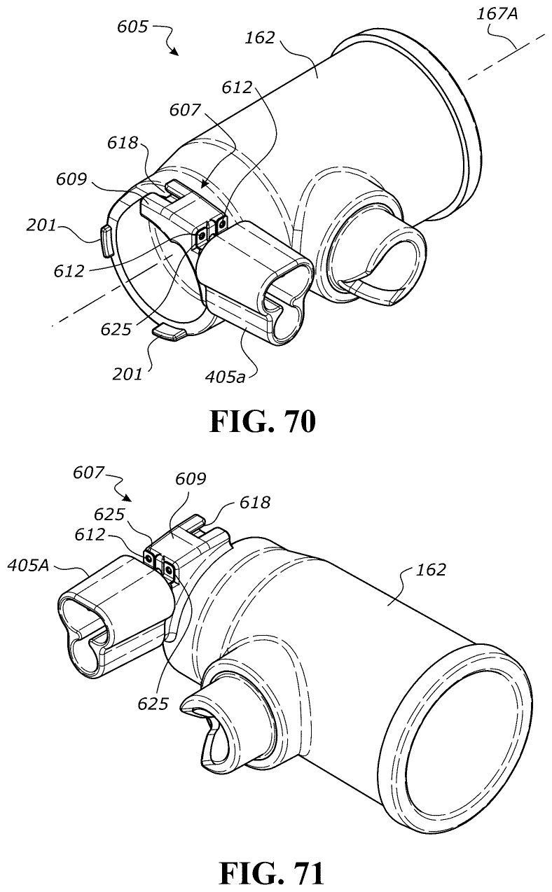

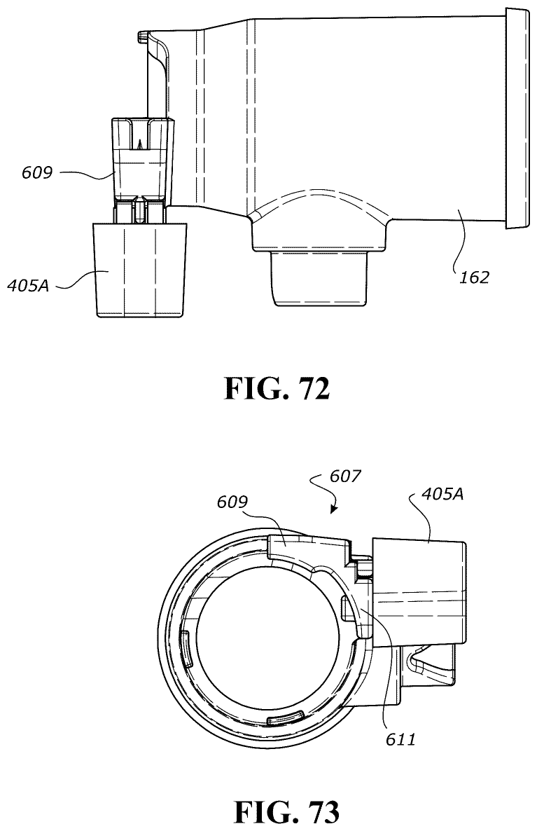

Referring to FIGS. 69 to 79 another device end connector 605 comprises similar features to the device end connector of FIGS. 23 to 26 and 29. The device end connector 605 may be similar to that described with reference to FIGS. 31-50.

The device end connector 605 comprises a connector body 162.

The connector body 162 may comprise one or more: overmould standoff(s) 201, and one or more probe port(s) 405B which locate one or more device end sensor(s) 411. The device end connector 605 may comprise a female distal end configured to receive a male part of the device.

The device end connector 405 comprises an electrical connector socket 405A for electrical connection via flying lead to the controller 125. Electrical connector socket 405A may be a separate rigid component, or may be integral with rigid connector 162. However, in the following, the electrical connector socket 405A is a separate component that is mounted onto the rigid connector 162.

The electrical connector socket 405A projects in a direction radially outwardly away from the lumen axis 167A.

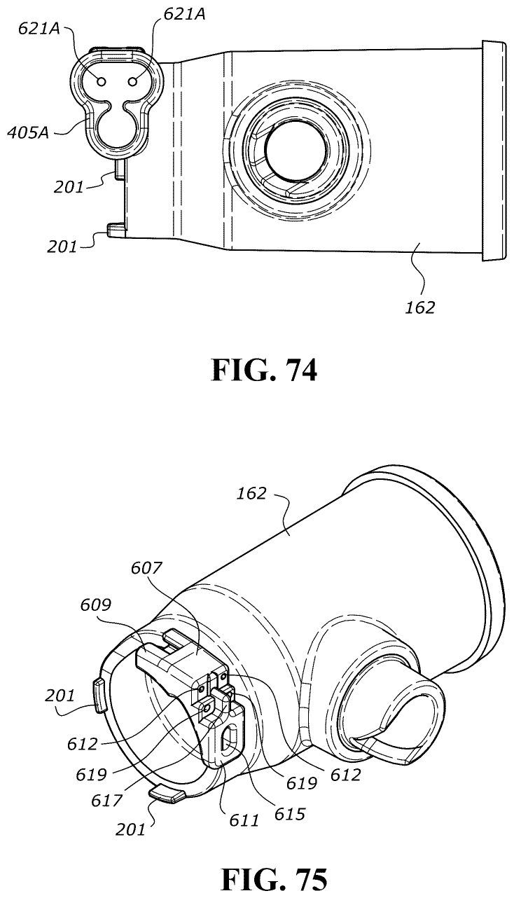

With reference to FIGS. 75 to 77, an electrical socket connector mount 607 is provided at one end of the rigid connector 162. The electrical socket connector 405a is configured to be mounted on mount 607.

Mount 607 is a separate component that is affixed to the rigid connector 162. For example, the mount 607 may comprise an arcuate base, the shape and dimensions of the arcuate base being configured to correspond to the outer arcuate surface of the rigid connector 162 so that the base of the mount 607 can rest on, and connect with, the outer surface of the rigid connector 162. The device end connector 605 therefore comprises three primary components: the rigid connector 162, the mount 607, and the electrical connector socket 405a.

Mount 607 may be integral with rigid connector 162 such that the rigid connector 162 and the mount 607 are a single component. The device end connector 605 may therefore comprise two primary components: the rigid connector/mount 162/607, and the electrical connector socket 405a.

In the embodiment of FIGS. 69-79, the mount 607 is located towards one end of the device end connector 605, for example in a position adjacent the end of the tube. The mount 607 may be mounted on the narrower, male end of the device end connector 605.

Mount 607 comprises a wire guide portion 609 and an electrical connector portion 611.

Wire guide portion 609 comprises a housing comprising an inlet opening 610 configured to receive the end of the bead 143, and the ends of the wires 151 that project from the end of the bead 143. The inlet opening 610 leads to an internal cavity of the housing. The housing captures and restrains the end of the bead 143.

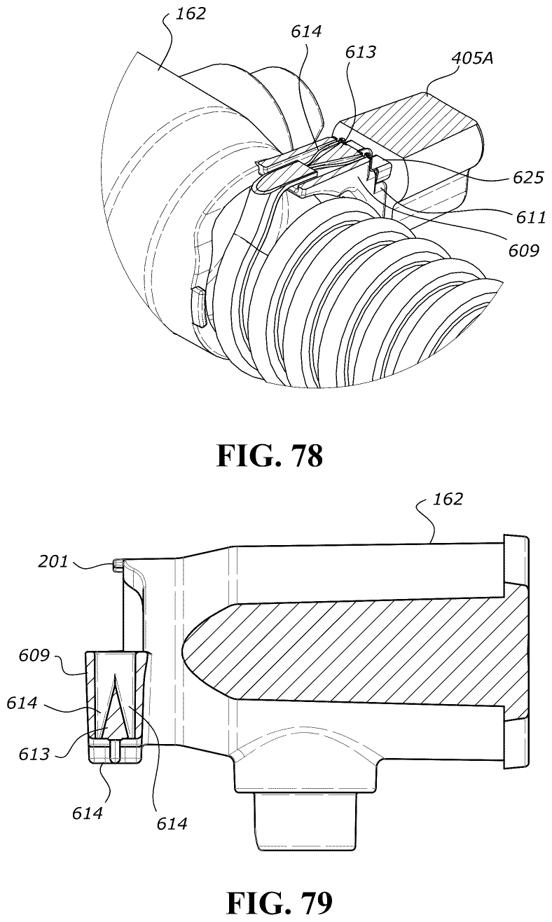

The housing comprises a pair of outlet openings 612 through which the ends of respective wires project. An internal wall comprising a wire deflector 613 is located inside the housing in between the outlet openings 612. The wire deflector 613 may be integral with the housing. The angled side faces 613A of the deflector 613 deflect the ends of the wires 151 toward respective outlet openings 612, as the bead 143 and wires 151 are inserted into the wire guide portion 609. When the bead 143 and wires 151 are fully inserted into the wire guide portion 609, the ends of the wires project from the outlet openings 612, as can best be seen in FIG. 78.

The wire deflector 613 may comprise a triangular block (when viewed from above, see FIG. 79), or may have a different configuration. For example, the wire deflector 613 may be arcuate, for example semi circular when viewed from above. The wire deflector 613 may comprise a straight, planar wall, extending along the centre of the housing.

With reference to FIGS. 78 and 79, the housing and wire deflector 613 define two wire guide channels 614 inside the housing, each channel 614 leading to a respective outlet opening 612. Thus, each wire 151 is located in its own channel 614, with the wire deflector 613 keeping the wires 151 separate such that the wires 151 do not contact each other, even if the wires 151 move during use or during cleaning (such as during autoclaving for example. During manufacture, overmould material may extend into the channels 614, over the wires 151, the overmould material extending all the way to the outlet openings 612.

The outlet openings 612 may comprise elongate bores through the end wall of the housing, so as to comprise portions of the wire guide channels 614 defined by the housing and wire deflector 613.

The housing further comprises an elongate slot that forms a window 618, through which the positions of the wires 151 can be seen during manufacture. This is help ensure that the manufacturer can see that the bead 143 is being retained. During manufacture the bead 143 can be pushed into the housing, and the manufacturer can see that the bead 143 has reached a terminal position. The bead 143 may protrude slightly out of the slot 618 in some embodiments.

The housing, adjacent the inlet opening 610, may be configured to receive the end of the bead 143. Thus, the wire guide portion 609 of the mount 607 may have a dual function to receive, locate and retain the end of the bead 143, and to receive, locate and retain the ends of the wires 151.

Electrical connector portion 611, is generally perpendicular to wire retention portion 609. The mount 607 comprises an arcuate underside 607A that mates with the arcuate outer surface of the connector body 162. The wire guide portion 609 extends tangentially in a first direction relative to the arcuate underside 607A. The electrical connector portion 611 extends tangentially in a second direction relative to the arcuate underside 607A.

Electrical connector portion 611 comprises mounting features that enable the electrical connector socket 405a to be mounted on the electrical connector portion 611. These mounting features include an elongate retention recess 615 configured to receive a corresponding protrusion on the electrical connector socket 405a, to correctly orientate and position the electrical connector socket 405a relative to the electrical connector portion 611, whilst retaining the two components together. The mounting features further comprise an elongate locating protrusion 617 configured to be received in a corresponding recess formed on the rear of the electrical connector socket 405a. The mounting features further comprise a pair of laterally spaced apart electrical connector pin retention recesses 619 configured to receive respective electrical pins 621, as will be described further below.

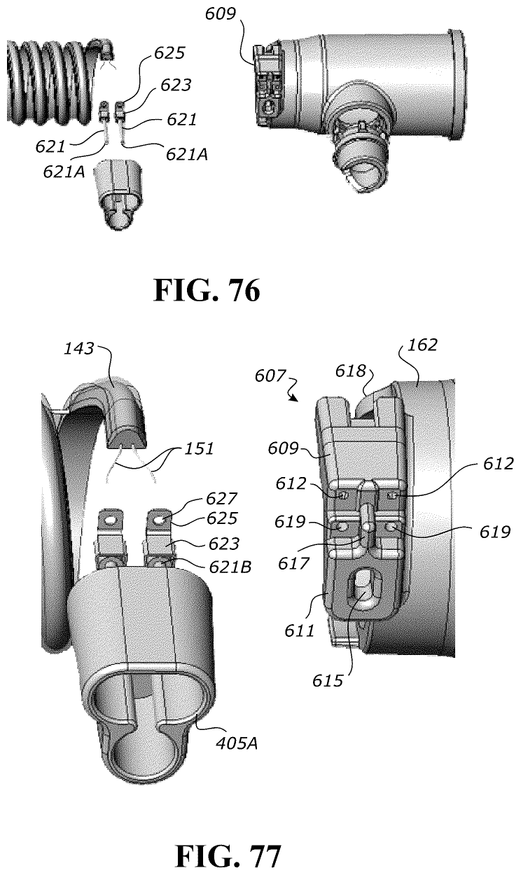

With additional reference to FIGS. 76 and 77, the electrical connection between the wires 151 and the electrical connector socket 405a is provided by intermediate electrical components located between the wire guide portion 609 and electrical connector portion 611. These intermediate electrical components comprise a pair of pins 621 the distal ends 621A of which project into the electrical connector socket 405a, and the proximal ends 621B of which are received in the pin retention recesses 619 of the electrical connector portion 611. A stepped bracket 623 is provided on each pin 621 between the distal and proximal ends 621A/B, each bracket 623 comprising an upwardly directed tab 625 comprising an aperture 627. Each aperture 627 is configured to receive an end of a respective wire 151, each wire end being received through apertures 627 and soldered in position to engage with the tabs 625 to retain each wire 151 on a respective bracket 623. Each tab 629 is dimensioned and orientated to abut the electrical connector portion 611 adjacent a respective wire outlet opening of the wire guide portion 609. When assembled, the brackets 623 form an electrically conductive path between the wires 151 and the pins 621. The pins 621 project into the electrical connector socket 405a, and can electrically connect with corresponding electrical connections on cable plug, the cable being connected to the device.

The rigid connector 162, the mount 607, and the electrical connector socket 405a, once so assembled, may be overmoulded.

A temperature sensor as used herein can be any suitable type of temperature sensor including, for example and without limitation, a thermistor, thermocouple, digital temperature sensor, transistor, and the like.

The parameters provided by or derived from the sensors can include, for example and without limitation, temperature, humidity, oxygen content, flow rate, or any combination of these or the like.

The controller 125 includes a power source configured to deliver electrical power to the heater wires. The power source can be a source of alternating current or direct current. In some embodiments, the controller 125 can receive input from a heater plate sensor. The heater plate sensor can provide the controller 125 with information regarding a temperature and/or power usage of the heater plate 131. In some embodiments, the controller 125 can receive input from a flow sensor 332. Any suitable flow sensor 332 can be used and the flow sensor 332 can be positioned between ambient air and the humidification chamber 129 or between the pressurized gas source 105 and the humidification chamber 129. In the illustrated system, the flow sensor 332 is positioned on the inlet port of the humidification chamber 129.

Aspects of the present disclosure are set out in the following paragraphs:

According to an aspect of this disclosure, we provide a connector for a medical tube comprising at least one wire, and a gases flow lumen extending through the connector;

the connector further comprising a wire retainer configured to retain an end portion of the wire on the connector, the wire retainer comprising a housing comprising a mouth configured to receive the end portion of the wire, and a cavity configured to enclose the end portion of the wire inside the housing.

According to an aspect of this disclosure, we provide a connector for a medical tube comprising at least one wire, the connector comprising a gases flow lumen extending through the connector;

the connector further comprising a wire retainer configured to retain an end portion of the wire on the connector, the wire retainer comprising a housing comprising a cavity having a longitudinal axis, and comprising a mouth configured to receive the end portion of the wire with the wire extending generally along the longitudinal axis into the cavity, the cavity comprising an end wall extending across the cavity in a direction across the longitudinal axis, the cavity being spaced from the mouth, the end wall being configured to limit movement of the end portion of the wire through the cavity in a direction along the longitudinal axis and away from the mouth.

According to an aspect of this disclosure, we provide a connector for a medical tube comprising at least one wire, the connector comprising a gases flow lumen extending through the connector, the connector further comprising a wire retainer comprising a housing configured to retain an end portion of the wire on the connector, the housing being configured to receive an end portion of the wire and to resist movement of the end portion of the wire in a direction along an x-axis of the connector, and in a direction along a y-axis of the connector, and in a direction along a z-axis of the connector.

According to an aspect of this disclosure, we provide a connector for a medical tube comprising at least one wire, the connector comprising a gases flow lumen extending through the connector and comprising a lumen axis, the connector further comprising a wire retainer configured to retain an end portion of the wire on the connector, the wire retainer comprising a housing configured to receive the end portion of the wire so as to restrain the end portion of the wire inside the housing in a direction along the connector and substantially parallel with the lumen of the connector, and in a direction radially outward and away from the lumen axis, and in a direction across the connector and tangentially outward and along the lumen axis.

The connector may comprise a first end configured to form part of a connection with the tube, and a second end configured to form part of a connection between the connector and another component.

The first end may be connected to the tube via an overmould that extends over at least part of the connector and over at least part of the tube.

The first end may be spaced from an end of the tube.

The connector may comprise a connector body defining the lumen, the wire retainer being mounted on the connector body.

The wire retainer may be integral with the connector body.

The wire retainer may project radially outwardly from the connector body.

The wire retainer may project longitudinally outwardly from the connector body.

The wire retainer may project from the first end of the connector.

The connector may be configured for use with a medical tube comprising a spirally wound bead, wherein the wire is encapsulated in the bead, wherein the wire retainer is configured to receive an end portion of the bead.

The wire retainer may electrically isolate the end portion of the wire when the end portion of the wire is received in the wire retainer.

The wire retainer may electrically isolate the end portion of the wire, that is, the wire retainer cannot electrically connect the end portion of the wire to another component.

The wire retainer may be configured such that the wire can only enter and exit the housing via the mouth.

The housing may comprise a cavity comprising a longitudinal axis.

The cavity longitudinal axis may be orthogonal to the longitudinal axis of the lumen.

The cavity may be configured to receive and retain the end portion of the wire such that the end portion of the wire is substantially orthogonal to the longitudinal axis of the lumen.

The cavity may comprise opposed ends, spaced along the longitudinal axis, a first opposed end comprising a mouth configured to receive the end portion of the wire.

A second opposed end of the cavity may comprise an end wall configured to resist further movement of the end portion of the wire through the housing in the direction of the longitudinal axis of the cavity.

The end wall may be shaped to provide a clearance at the end of the cavity to accommodate some or all of the end portion of the wire.

The end wall may be arcuate.

The wire retainer may comprise an inclined guide surface configured to guide the end portion of the bead and/or the end portion of the wire into the cavity of the elongate housing.

The cavity may comprise opposed side walls extending substantially parallel to the longitudinal axis, the side walls resisting lateral movement of the wire away from the longitudinal axis.

The side walls may be parallel.

The side walls may be inclined.

The cavity may comprise a base wall, the base wall resisting movement of the wire away from the longitudinal axis towards the lumen.

The base wall may extend substantially parallel to the longitudinal axis

The cavity may comprise a ceiling, the ceiling resisting movement of the wire away from the longitudinal axis, away from the lumen.

The ceiling of the cavity may extend substantially parallel to the longitudinal axis.

The top may be arcuate, or may comprise at least one arcuate portion, for example when viewed along the longitudinal axis of the cavity.

An intersection between a side wall and the top of the cavity may be arcuate.

The cavity may be cuboidal.

The cavity may be configured such that the wire can only enter and exit the cavity via the mouth.

The cavity may comprise a plurality of cavity portions of different sizes.

The cavity may comprise a larger cavity portion configured to receive and contain an end of a bead of the medical tube, and a smaller cavity portion configured to receive and contain the end portion of the wire.

The large cavity portion may be positioned further from the lumen than the smaller housing portion.

The cavity may comprise a further cavity portion configured to provide a clearance between the bead and the cavity.

The lumen may comprise a lumen axis.

The longitudinal axis of the cavity may be non-aligned with the lumen axis.

The longitudinal axis of the cavity may be perpendicular to the lumen axis.

The longitudinal axis of the cavity may extend laterally across the connector.

The wire retainer may be radially outwardly spaced from the lumen, such that there is a clearance between an underside of the wire retainer and the lumen, when viewed along the lumen axis.

The lumen may comprise an arcuate profile when viewed along the lumen axis.

An external portion of the housing may be arcuate so as to correspond to at least part of the arcuate profile of the lumen.

The longitudinal axis of the cavity may extend tangentially to the arcuate profile of the lumen.

The end portion of the wire may extend in a direction perpendicular to the lumen axis, when the end portion of the wire is received in the cavity.

According to an aspect of this disclosure there is provided a medical tube and the connector of any one of the preceding statements.

The medical tube may comprise a helically wound bead extending along the tube. The, or a, wire may be encapsulated in the bead.

The longitudinal axis of the cavity may be substantially parallel with the longitudinal axis of an end of the bead.

The connector may comprise an overmould, the connector body being at least partially connected to the medical tube using the overmould.

According to another aspect of this disclosure there is provided a breathing circuit comprising:
the connector of any one of the preceding statements;
an inspiratory tube; and
at least one further medical tube.

The at least one further medical tube may comprise at least one wire.

According to an aspect of this disclosure there is provided a segmented inspiratory limb for transporting a humidified gas, comprising:
a) a first tube segment comprising a first lumen for transporting the humidified gas, and a first heater wire;
b) a second tube segment comprising a second lumen for transporting the humidified gas, and a second heater wire;
   wherein at least one of the first and/or second lumens is at least partially formed from a spirally wound member.

The spirally wound member may be elongate and may comprise a hollow body spirally wound to form at least in part the lumen, and a hollow wall surrounding the lumen.

At least one tube segment may comprise a second elongate member spirally wound and joined between adjacent turns of the helically wound member, the second elongate member forming at least a portion of the lumen of the elongate tube.

According to an aspect of this disclosure there is provided a respiratory humidification system comprising:
a humidifier to humidify a breathable gas to create humidified gas;
an inspiratory limb comprising:
   a first tube segment comprising a first lumen for transporting the humidified gas from the humidifier, and a first heater wire;
   a second tube segment comprising a second lumen for transporting the humidified gas from the first tube segment, and a second heater wire; and
   an intermediate connector comprising a connector lumen, and configured to pneumatically connect the first tube segment to the second tube segment; and to electrically connect the first and second heater wires;
a first sensor positioned at a humidifier-end of the first segment, and a second sensor positioned at the intermediate connector; and
a controller:
   wherein the controller is adapted to provide electrical power to the first heater wire based on a first sensor output from the first sensor and a second sensor output from the second sensor, the first heater wire providing electrical power to the second heater wire.

According to an aspect of this disclosure there is provided a surgical humidification system comprising:
a humidifier to humidify a breathable gas to create humidified gas;
a surgical gases delivery limb comprising:
   a first tube segment comprising a first lumen for transporting the humidified gas from the humidifier, and a first heater wire;
   a second tube segment comprising a second lumen for transporting the humidified gas from the first tube segment, and a second heater wire; and
   an intermediate connector comprising a connector lumen, and configured to pneumatically connect the first tube segment to the second tube segment; and to electrically connect the first and second heater wires;
a first sensor positioned at a humidifier-end of the first segment, and a second sensor positioned at the intermediate connector; and
a controller;
   wherein the controller is adapted to provide electrical power to the first heater wire based on a first sensor output from the first sensor and a second sensor output from the second sensor, the first heater wire providing electrical power to the second heater wire.

In this embodiment, both the first and second heater wires may be controlled to receive electrical power and to simultaneously provide heating to the first and second tube segments respectively.

The first heater wire may be controlled via the controller using closed loop control based on feedback from the first and/or second sensors.

The second heater wire may be controlled using open loop control.

In this embodiment, the second tube end may comprise a first end for connection to the intermediate connector, and a second end which is distal from the first end, wherein the second end is not provided with a sensor.

An inspiratory limb comprising:
a first tube segment comprising a first lumen for transporting humidified gas from a humidifier:
   and a first heater wire;
a second tube segment comprising a second lumen for transporting the humidified gas from the first tube segment, and a second heater wire; and
an intermediate connector comprising a connector lumen, and configured to pneumatically connect the first tube segment to the second tube segment; and to electrically connect the first and second heater wires; and
a first sensor positioned at a humidifier-end of the first segment, and a second sensor positioned at the intermediate connector.

Certain terminology may be used in the description for the purpose of reference only, and thus are not intended to be limiting. For example, terms such as “above” and “below” refer to directions in the drawings to which reference is made. Terms such as “front,” “back,” “left,” “right,” “rear,” and “side” describe the orientation and/or location of portions of the components or elements within a consistent but arbitrary frame of reference which is made clear by reference to the text and the associated drawings describing the components or elements under discussion. For example, as the context may dictate, the terms “front” and/or forward can be used relative to components described herein positioned relatively or entirely distal to the user's face when the mask assembly as described herein is worn by the user. As the context may dictate, the terms “rear” and/or “back” can be used relative to components described herein positioned relatively or entirely proximal to the user's face and/or components that are forward or at the front of the mask assembly when the mask assembly as described herein is worn by the user. Moreover, terms such as “first,” “second,” “third,” and so on may be used to describe separate components. Such terminology may include the words specifically mentioned above, derivatives thereof, and words of similar import.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”. Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

The term “plurality” refers to two or more of an item. Recitations of quantities, dimensions, sizes, formulations, parameters, shapes and other characteristics should be construed as if the term “about” or “approximately” precedes the quantity, dimension, size, formulation, parameter, shape or other characteristic. The terms “about” or “approximately” mean that quantities, dimensions, sizes, formulations, parameters, shapes and other characteristics need not be exact, but may be approximated and/or larger or smaller, as desired, reflecting acceptable tolerances, conversion factors, rounding off, measurement error and the like and other factors known to those of skill in the art. Recitations of quantities, dimensions, sizes, formulations, parameters, shapes and other characteristics should also be construed as if the term “substantially” precedes the quantity, dimension, size, formulation, parameter, shape or other characteristic. The terms “approximately,” “about,” and “substantially” as used herein represent an amount close to the stated amount that still performs a desired function or achieves a desired result. For example, in some embodiments, as the context may dictate, the terms “approximately”, “about”, and “substantially” may refer to an amount that is within less than or equal to 10% of the stated amount. The term “generally” as used herein represents a value, amount, or characteristic that predominantly includes, or tends toward, a particular value, amount, or characteristic. For example, as the context may dictate, the term “generally linear” can mean something that departs from exactly parallel by less than or equal to 15°.

Numerical data may be expressed or presented herein in a range format. It is to be understood that such a range format is used merely for convenience and brevity and thus should be interpreted flexibly to include not only the numerical values explicitly recited as the limits of the range, but also interpreted to include all of the individual numerical values or sub-ranges encompassed within that range as if each numerical value and sub-range is explicitly recited. As an illustration, a numerical range of “1 to 5” should be interpreted to include not only the explicitly recited values of about 1 to about 5, but should also be interpreted to also include individual values and sub-ranges within the indicated range. Thus, included in this numerical range are individual values such as 2, 3 and 4 and sub-ranges such as “1 to 3,” “2 to 4” and “3 to 5,” etc. This same principle applies to ranges reciting only one numerical value (e.g., “greater than 1”) and should apply regardless of the breadth of the range or the characteristics being described.

A plurality of items may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. Furthermore, where the terms “and” and “or” are used in conjunction with a list of items, they are to be interpreted broadly, in that any one or more of the listed items may be used alone or in combination with other listed items. The term “alternatively” refers to selection of one of two or more alternatives, and is not intended to limit the selection to only those listed alternatives or to only one of the listed alternatives at a time, unless the context clearly indicates otherwise.

Reference to any prior art in this specification is not, and should not be taken as, an acknowledgement or any form of suggestion that that prior art forms part of the common general knowledge in the field of endeavour in any country in the world.

The disclosure may also be said broadly to consist in the parts, elements and features referred to or indicated in the specification of the application, individually or collectively, in any or all combinations of two or more of said parts, elements or features.

Where, in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the disclosure and without diminishing its attendant advantages. For instance, various components may be repositioned as desired. It is therefore intended that such changes and modifications be included within the scope of the disclosure. Moreover, not all of the features, aspects and advantages are necessarily required to practice the present disclosure. Accordingly, the scope of the present disclosure is intended to be defined only by the claims that follow.

1-57. (canceled)

58. A medical tube for transporting a humidified gas; comprising
a first tube segment comprising a first lumen for transporting the humidified gas, and a first heater wire;
a second tube segment comprising a second lumen for transporting the humidified gas, and a second heater wire; wherein
the first tube segment is configured to be electrically connected to, and to use electrical power provided by, a power source; and
wherein the second heater wire also uses the electrical power; wherein
the first heater wire has a higher resistance value than that of the second heater wire.

59-65. (canceled)

66. The medical tube of claim 58, wherein the first heater wire and/or the second heater wire comprises a property that determines, or partially determines the electrical power used by the first heater wire and/or the second heater wire; wherein the second tube segment is downstream of the first tube segment, and wherein the property of the first tube segment and/or the second tube segment are configured such that between 3-20%, and optionally 3-12%, of a total electrical power delivered to the first heater wire and the second heater wire by the power source is used by the second tube segment.

67. The medical tube of claim 58 comprising any one or more of the following characteristics:
a) a length of the first tube segment is between 1100-1400 mm, optionally 1300 mm;
b) a length of the second tube segment is between 200-400 mm, optionally 300 mm;
c) a diameter of the first heater wire in the first tube segment is between 0.1 and 0.3 mm, optionally approximately 0.2 mm;
d) a diameter of the second heater wire in the second tube segment is between 0.2 and 0.5 mm, and optionally approximately 0.3 mm;
e) a total resistance of the first heater wire and the second heater wire is between 10 and 25Ω, and optionally is approximately 17Ω;
f) a resistance of the first heater wire of the first tube segment is between 10 and 25Ω, and optionally is approximately 15Ω;
g) a resistance of the second heater wire of the second tube segment is between 0.5 and 3Ω, and optionally is approximately 1Ω.

68-75. (canceled)

76. The medical tube of claim 58, wherein the first tube segment and the second tube segment are electrically connected in series.

77-79. (canceled)

80. The medical tube of claim 76 further comprising an intermediate connector, wherein the first lumen and the second lumen are connected together via the intermediate connector.

81. The medical tube of claim 80, wherein the intermediate connector comprises a probe port body comprising a probe port configured to receive a probe to measure a property of the humidified gas, wherein the probe comprises a temperature sensor and/or a flow sensor.

82. (canceled)

83. The medical tube of claim 81 comprising a device end connector configured to connect the first tube segment to a device providing a flow of the humidified gas and/or wherein the device end connector comprises an electrical connector configured to be connected to a device comprising the power source.

84-85. (canceled)

86. The medical tube of claim 81, wherein the intermediate connector comprises a rigid connector body comprising a lumen for transporting the humidified gas, the lumen comprising a longitudinally extending lumen axis.

87. The medical tube of claim 86, wherein the intermediate connector comprises an overmould that encapsulates some or all of the rigid connector body.

88. The medical tube of claim 87, wherein the overmould extends beyond at least one end of the rigid connector body and/or wherein the overmould extends over the first tube segment and/or the second tube segment.

89. (canceled)

90. The medical tube of claim 87, wherein the overmould pneumatically connects the first tube segment and/or the second tube segment to the rigid connector body.

91. The medical tube of claim 87, wherein the overmould extends at least partially over the probe port body.

92-95. (canceled)

96. The medical tube of claim 86, wherein one of the first heater wire and the second heater wire is wound around the rigid connector body in a clockwise direction, and the other of the first heater wire and the second heater wire being wound around a periphery of the rigid connector body in an anti-clockwise direction, when the rigid connector body is viewed from a first end of the rigid connector body along a longitudinal axis extending through the lumen of the rigid connector body.

97. The medical tube of claim 96 wherein the first heater wire and the second heater wire extend around between 25% and 75% of the periphery of the rigid connector body, when viewed along the longitudinal axis.

98. The medical tube of claim 97 wherein at least one of the first tube segment and the second tube segment comprises a spirally wound body, wherein at least one of the first heater wire and the second heater wire, or at least a portion thereof, is internal of the spirally wound body, so as to be at least partially encapsulated by the spirally wound body.

99-123. (canceled)

124. A medical tube for transporting a humidified gas, the medical tube comprising:
a first tube segment comprising a first lumen for transporting the humidified gas, the first tube segment also comprising a first heater wire;
a second tube segment comprising a second lumen for transporting the humidified gas, the second tube segment also comprising a second heater wire;
wherein the first heater wire is configured to be electrically connected to, and to use electrical power provided by, a power source, the second heater wire also using the electrical power provided by the power source, and
wherein a proportion of a total electrical power used by the second heater wire is between 3 and 20% of a total electrical power used by the first tube segment and the second tube segment.

125. A medical tube for transporting a humidified gas, the medical tube comprising:
a first tube segment comprising a first lumen for transporting the humidified gas, the first tube segment also comprising a first heater wire;
a second tube segment comprising a second lumen for transporting the humidified gas, the second tube segment also comprising a second heater wire;
the first tube segment being configured to be electrically connected to, and to use electrical power provided by, a power source; and
the second heater wire also uses the electrical power and the first heater wire determines the electrical power used by the second heater wire.
